(19) United States
(12) Patent Application Publication
(10) Pub. No.: US 2011/0152210 A1
(43) Pub. Date: Jun. 23, 2011

(54) POLYPHENOL COMPOUNDS FOR INHIBITING PROTEASOME AND USES THEREOF

(75) Inventors: Tak-Hang CHAN, Toronto (CA); Q. Ping DOU, Grosse Pointe, MI (US)

(73) Assignees: THE ROYAL INSTITUTION FOR THE ADVANCEMENT OF LEARNING/McGILL UNIVERSITY, Montreal (CA); THE HONG KONG POLYTECHNIC UNIVERSITY, SAR (CN); WAYNE STATE UNIVERSITY, Detroit, MI (US)

(21) Appl. No.: 12/968,759

(22) Filed: Dec. 15, 2010

Related U.S. Application Data
(60) Provisional application No. 61/288,014, filed on Dec. 18, 2009.

(51) Int. Cl. A61K 31/235; C07C 69/94; C07C 229/48; A61K 31/24; C12N 5/071; C12N 9/99; C07C 67/08; C07C 227/02; A61K 31/69; A61K 31/337; A61K 31/437; A61K 31/7048; A61K 31/407; A61K 31/352; A61P 35/00; A61P 35/02

(52) U.S. Cl. 514/27; 560/70; 560/65; 560/66; 560/49; 560/83; 514/533; 435/375; 435/184; 514/64; 514/449; 514/283; 514/421; 514/456

(57) ABSTRACT

Synthetic polyphenolic compounds of formula (I), their modes of synthesis, and pharmaceutical compositions thereof are provided herein. Use of the compounds and compositions described herein for inhibiting proteasomal activity and for treating cancer is also provided.

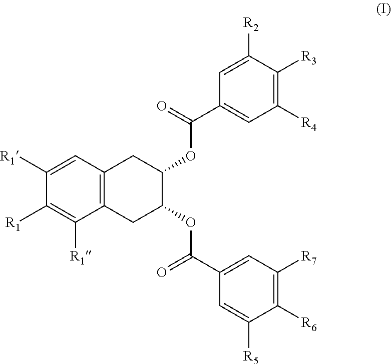

### CROSS-REFERENCE TO RELATED APPLICATIONS

This application claims priority to U.S. provisional application No. 61/288,014 filed Dec. 18, 2009, the entire contents of which are hereby incorporated by reference.

### FIELD OF THE INVENTION

This invention relates to novel compounds and compositions comprising analogs of epigallocatechin gallate, particularly for use as proteasome inhibitors and for treating cancer.

### BACKGROUND OF THE INVENTION

The ubiquitin-proteasome system (UPS) is responsible for the highly regulated degradation of intracellular proteins having important roles in cellular functions (Hershko, A. Cell Death Differ. 12, 1191 (2005)). One compound that targets the UPS is the proteasome inhibitor bortezomib (Velcade™), which is used clinically for the treatment of patients with multiple myeloma or mantle cell lymphoma. Velcade™ is an N-substituted dipeptidyl boronic acid. Another proteasome inhibitor is salinosporamide, a marine natural product characterized by a functionalized β lactone (Feling R H, et al., (2003) Angew. Chem. Int. Ed. Engl. 42(3): 355-7). Yet another inhibitor of the proteasome is epigallocatechin gallate (EGCG) and its analogs (U.S. Pat. No. 7,358,383 B2; U.S. Pat. No. 6,713,506; US 2008/0015248 A1; WO 2006/017981; Landis-Piwowar, K. R et al., Cancer Res. (2007), 67, 4303; Lee, S. K. et al., Nutri. Cancer (2008), 60, 483).

Proteasomes are large multi-catalytic protease complexes responsible for degrading the majority of cellular proteins. The 20S-core particle of the 26S proteasome is a barrel-shaped superstructure, and the sites of proteolytic activity reside in the interior.

The eukaryotic proteasome is known to have proteolytic activity that is associated with its β subunits. For example, the β5 subunit is associated with chymotrypsin-like proteolytic activity (cleavage after hydrophobic residues); the β2 subunit exhibits trypsin-like activity (cleavage after basic residues); and the β1 subunit is responsible for caspase-like activity (cleavage after acidic residues). These three proteolytic properties depend on the presence of an N-terminal Threonine (Thr 1) residue. The hydroxyl group of the Thr 1 side chain is responsible for catalyzing cleavage of substrate peptides through nucleophilic attack. Binding pockets adjacent to the N-terminal Threonine residue recognize the side chains of substrate peptides to be degraded and confer upon each catalytic site its substrate specificity. The S1 pocket of the β5 subunit is defined by hydrophobic residues, Ala 20, Val 31, Ile 35, Met 45, Ala 49, and Glu 53, and this binding pocket has been shown to be important for substrate specificity and binding of several types of proteasome inhibitors (Smith, D. M. et al., Proteins: Structure, Function, and Bioinformatics (2004) 54, 58; Dou, Q. P. et al., Inflammopharmacology (2008) 16, 208).

Catechol-O-methyl transferase (COMT) is an enzyme widely distributed throughout the body (Mannisto, P. T. and Kaakkola, S., Pharmacol Rev. (1999) 51, 593). Certain endogenous catecholamine neurotransmitters, such as dopamine, noradrenaline and adrenaline, as well as the amino acid L-DOPA and also catecholestrogens are substrates of COMT.

COMT is also able to methylate one or more of the phenolic groups of (−)-EGCG (Zhu, B. T. et al., Drug Metab. Dispos. (2000) 28, 1024; Meng, X. et al. Chem. Res. Toxicol. (2002) 15, 42). In humans, a single gene for COMT encodes both a soluble COMT (S-COMT) and a membrane-bound COMT (MB-COMT).

A single nucleotide polymorphism (G to A) in codon 108 (S-COMT) or 158 (MB-COMT) results in a valine to methionine (Val to Met) substitution, leading to a high-(Val/Val [H/H]), intermediate-(Val/Met [H/L]), or low-activity (Met/Met [L/L]) form of COMT (Lachman, H. M. et al., Pharmacogenetics. (1996) 6, 243.). There is a three-to-four-fold difference in enzyme activity between the high- and low-activity expressed genes (Weinshilboum, R. M. et al., Annu Rev Pharmacol Toxicol. (1999) 39, 19). A recent case-control study of breast cancer in Asian-American women revealed that women who consumed green tea and who also carried the low activity COMT polymorphism had a reduced risk of breast cancer (Wu, A. H. et al., Cancer Res. (2003) 63.7526). In contrast, among those who were homozygous for the high activity COMT allele, breast cancer risk did not differ between tea drinkers and non-tea drinkers. These data suggest that EGCG and other tea polyphenols may be less cancer-protective upon methylation.

There is a need to provide analogs of (−)-EGCG that are able to overcome at least one, but preferably more, of the problems as set forth in the prior art. It would be desirable to provide compounds that are able to overcome the limitations of (−)-EGCG in cancer therapy, as well as to provide polyphenols that inhibit the proteasome and/or that are not as prone to deactivation through methylation by COMT compared to (−)-EGCG and other polyphenols of the prior art.

### SUMMARY OF THE INVENTION

Novel compounds and compositions and methods of use thereof for treating cancer and/or inhibiting proteasomal activity in a cell are provided.

In accordance with an embodiment of the invention, there are provided herein compounds of the formula (I):

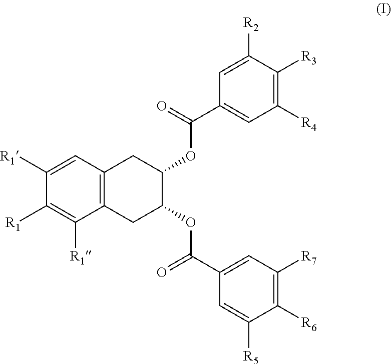

wherein R₁, R₁′ and R₁″ are each independently selected from the group consisting of H, alkyl, alkenyl, cycloalkyl, heterocycloalkyl, cycloalkenyl, heterocycloalkenyl, aryl, halogen, OH, an acyloxy group, and NR₈,R₉, wherein R₈ and R₉ are independently selected from the group consisting of H, alkyl, alkenyl, cycloalkyl, heterocycloalkyl, cycloalkenyl, heterocycloalkenyl, aryl, and acyl, any of which may be optionally substituted;
R₂, R₄, R₅ and R₇ are each independently H, alkyl, alkenyl, cycloalkyl, heterocycloalkyl, cycloalkenyl, heterocycloalkenyl, aryl, OH, acyloxy or halogen; and
R₃ and R₆ are each independently H, OH, acyloxy, NR₈R₉ or a halogen, wherein R₈ and R₉ are as defined above;
and analogs thereof; and pharmaceutically acceptable salts thereof.

In accordance with another embodiment of the present invention there are provided compounds of the formula (Ia):

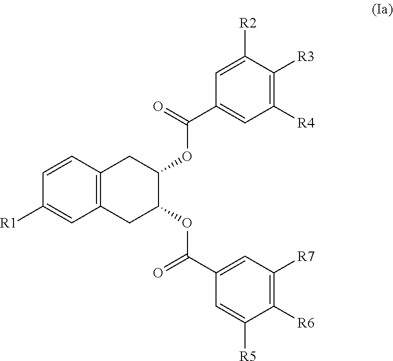

wherein R₁ is selected from the group consisting of H, alkyl, alkenyl, cycloalkyl, heterocycloalkyl, cycloalkenyl, heterocycloalkenyl, aryl, halogen, OH, an acyloxy group, and NR₈,R₉, wherein R₈ and R₉ are independently selected from the group consisting of H, alkyl, alkenyl, cycloalkyl, heterocycloalkyl, cycloalkenyl, heterocycloalkenyl, aryl, and acyl, any of which may be optionally substituted;
R₂, R₄, R₅ and R₇ are each independently H, alkyl, alkenyl, cycloalkyl, heterocycloalkyl, cycloalkenyl, heterocycloalkenyl, aryl, OH, acyloxy or halogen; and
R₃ and R₆ are each independently H, OH, acyloxy, NR₈R₉ or a halogen, wherein R₈ and R₉ are as defined above;
and analogs thereof; and pharmaceutically acceptable salts thereof.

In another embodiment of the invention there are provided compounds of formula (I) or (Ia), wherein R₁ is selected from the group consisting of H, halogen, OH, and an acyloxy group; R₂, R₄, R₅ and R₇ are each independently H, alkyl, alkenyl, cycloalkyl, heterocycloalkyl, cycloalkenyl, heterocycloalkenyl, aryl, OH, acyloxy or halogen; and R₃ and R₆ are each independently H, OH, acyloxy, NR₈R₉ or a halogen; and analogs thereof; and pharmaceutically acceptable salts thereof.

In accordance with another embodiment of the present invention, there is provided a compound having the structure of formula II:

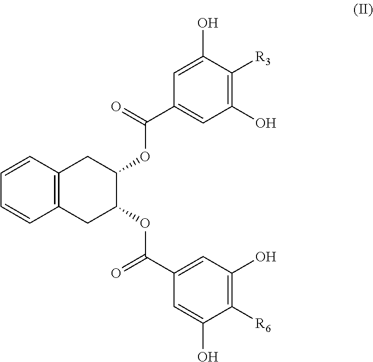

wherein:
R₃ and R₆ are both H, Br, F, Cl or CH₃;
or an analog thereof; and pharmaceutically acceptable salts thereof.

In accordance with yet another embodiment of the present invention, there is provided a compound having the structure of formula III:

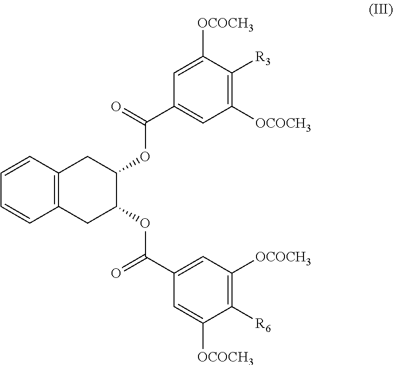

wherein:
R₃ and R₆ are both OCOCH₃, H, Br, F, Cl or CH₃;
or an analog thereof; and pharmaceutically acceptable salts thereof.

In accordance with yet another embodiment of the present invention, there is provided a compound having the structure of formula IV:

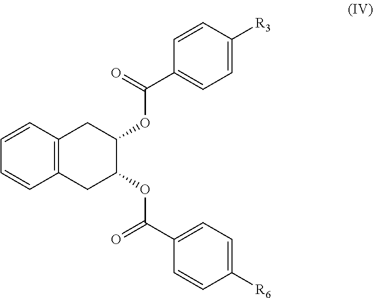

wherein:
R₃ and R₆ are both OH, OCOCH₃, NHCOOC(CH₃)₃, NH₂ or NHCOCH₃;
or an analog thereof;
and pharmaceutically acceptable salts thereof.

In another embodiment of the invention, there is provided a compound having the structure of formula V:

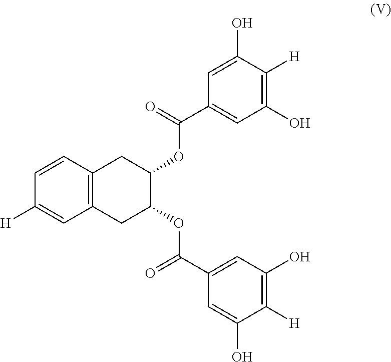

or an analog thereof; and pharmaceutically acceptable salts thereof.

In another embodiment of the invention, there is provided a compound having the structure of formula VI:

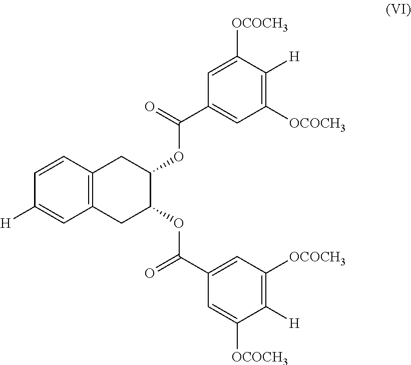

or an analog thereof; and pharmaceutically acceptable salts thereof.

In another embodiment of the invention, there is provided a compound having the structure of formula VII:

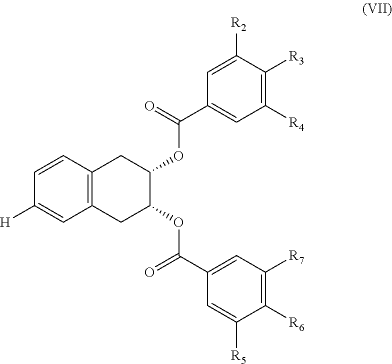

wherein R₂, R₃, R₄, R₅, R₆ and R₇ are F; or

### R₂, R₃, R₅ and R₆ are F, and R₄ and R₇ are H; or

### R₂, R₄, R₅ and R₇ are F, and R₃ and R₆ are H;

or an analog thereof;
and pharmaceutically acceptable salts thereof.

In another embodiment of the invention, there are provided compounds having the structure of formulae VIII, IX and X:

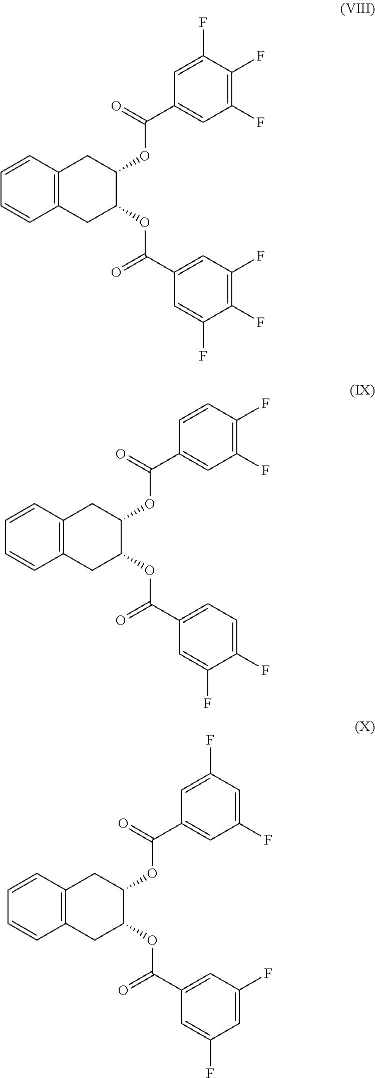

or an analog thereof; and pharmaceutically acceptable salts thereof.

In another embodiment, there are provided herein compounds having the structures described herein, for example the structures shown in Table 1, Scheme 1, Scheme 2, and Scheme 3, and analogs and pharmaceutically acceptable salts thereof. In one embodiment, the compound of the invention is an analog of a tea polyphenol. In an embodiment, the compound of the invention is an EGCG analog.

In another embodiment, there are provided herein pharmaceutical compositions comprising at least one compound as provided herein and a pharmaceutically acceptable carrier. In an embodiment, the pharmaceutical compositions further comprise at least one additional active ingredient or therapeutic agent. For example, the pharmaceutical compositions may further comprise a second agent which is an anti-cancer therapeutic, a chemotherapeutic agent, or a proteasomal inhibitor, such as bortezomib (Velcade™), Taxol™, docetaxel, vinblastine, vincristine, camptothecin toptecan, etoposid, teniposide, salinosporamide, epigallocatechin gallate or an analog thereof.

Also provided herein are methods for inhibiting proteasomal activity in a cell, comprising contacting the cell with an effective amount of at least one compound or pharmaceutical composition of the invention, such that proteasomal activity in the cell is inhibited. The contacting may occur in vitro or in vivo. Compounds and compositions may be administered by a variety of routes, such as orally, parenterally, subcutaneously, intravenously, intramuscularly, intraperitoneally, intraarterially, transdermally, and via mucosal administration. In an aspect, the proteasome may be a 20S proteasome or a 26S proteasome. In a further aspect, the chymotrypsin activity and/or the chymotrypsin-like activity of the 20S proteasome is inhibited.

There is further provided herein a method for treating cancer in a subject, e.g. a human, comprising administering a therapeutically effective amount of at least one compound or composition of the invention to the subject. In an aspect, cancer cell growth is inhibited in the subject, cancer cell apoptosis is induced in the subject, and/or proteasomal activity is inhibited in the subject. The cancer may be, for example, prostate cancer, leukemia, hormone dependent cancer, breast cancer, colon cancer, lung cancer, epidermal cancer, liver cancer, esophageal cancer, stomach cancer, cancer of the brain, multiple myeloma and/or kidney cancer.

Methods for synthesizing the compounds described herein are also provided. In an aspect, dihydronaphthalene is reacted with osmium tetroxide, followed by acylation with two or more equivalents of a substituted protected aryl benzoic acid and a dehydrating agent, removal of benzyloxy protecting group in the presence of a catalyst, and optionally reacting the compound with an acylating agent.

### BRIEF DESCRIPTION OF THE DRAWINGS

Particular embodiments of the present invention will now be explained by way of example and with reference to the accompanying drawings, in which:

FIG. 1 shows the structures of the (−)-EGCG, its analogs and derivative Pro-EGCG. The nomenclature of the rings is used throughout this specification.

FIG. 2 shows the proteasome inhibition by (−)-EGCG and its analogs. Purified 20S proteasome was incubated with compound 5, 7, 16, or 21 (A) or MDA-MB-231 cell extract (5.7 μg) was incubated with compound 6, 8, 17, or 22 (B) at indicated concentrations for 2 hours, followed by measuring the proteasomal chymotrypsin-like activity. EGCG was used as a comparative standard.

FIG. 3 shows the inhibition of cellular proteasome by EGCG and EGCG analogs. (A) MDA-MB-231 cell extracts (5.7 μg) were incubated with different concentrations of compound 5 or 7 or EGCG for 2 hours, followed by performance of proteasomal chymotrypsin-like activity assay. EGCG was used as a control. (B) MDA-MB-231 cell extracts (5.7 μg) were pre-treated with 10 μM DNC for 20 minutes, followed by co-incubation with 5, or 7 or EGCG for 2 h. The proteasomal chymotrypsin-like activity was measured.

FIG. 4 shows the inhibition of cell proliferation by EGCG analogs. Human breast cancer MDA-MB-231 cells were treated with 25 or 50 μM EGCG analogs for 24 hours, followed by MTT assay. Pro-EGCG was used as a comparison.

FIG. 5 shows the effects of DNC on EGCG analogs efficacy against cell proliferation. Human breast cancer MDA-MB-231 cells with high COMT activity were treated with 50 μM EGCG analogs for 24 hours in the absence or presence of 10 μM DNC, followed by MTT assay. Pro-EGCG was used as a comparison.

FIG. 6 shows the accumulation of proteasome substrates upon contacting MDA-MB-231 cells with analogs 6, 8 and Pro-EGCG. MDA-MB-231 cells were treated with 50 μM EGCG analogs for 22 hours. Extracted proteins were subject to Western blotting analysis using antibodies against ubiquitinated proteins and actin.

FIG. 7 shows the effect of compounds 5, 7 and 23 on human multiple myeloma ARP cells (A) or OPM1 cells (B). The cells were treated with Velcade alone or with 20 μM of compounds 5, 7 and 23 or in combination with varying doses of Velcade for 48 hrs, followed by a MTT assay.

FIG. 8 shows color changes of MTT assay in a 96 well-plate in the same experiment as FIG. 7A. Deep purple color indicates fully viable cells; light purple color indicates a reduced number of viable cells; and yellowish color indicates an absence of viable cells.

### DETAILED DISCLOSURE OF THE INVENTION

The present invention is directed to polyphenolic compounds useful for inhibiting proteasomal activity, methods of synthesis thereof, pharmaceutical compositions thereof, and use thereof for proteasome inhibition and for treating cancer. In particular, the polyphenol compounds of the present invention inhibit the chymotrypsin-like activity of a proteasome. The polyphenol compounds of the present invention may be synthesized using methods disclosed herein.

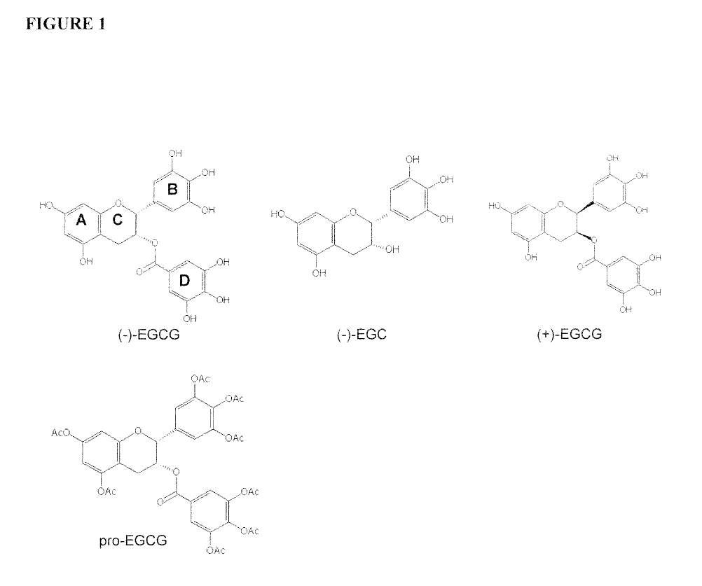

The nomenclature of FIG. 1, whereby the rings of (−)-EGCG are named A, B, C or D, is utilized throughout the specification.

One embodiment of the subject invention is directed to polyphenolic compounds having a similar ring structure to green tea polyphenols. More particularly, in an embodiment the compounds of the present invention possess an adequate number of phenol substituents or carbonyl oxygens to ensure favorable binding and inhibition of the proteasome. In an embodiment, analogs of green tea polyphenols are provided.

In accordance with another embodiment of the invention, the polyphenol analogs disclosed herein are symmetrical and do not contain the phenolic substitution pattern of epigallocatechin or epigallocatechin gallate.

In accordance with another embodiment of the present invention, there is provided a compound having the structure of formula I:

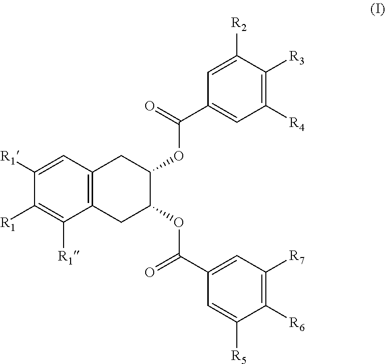

wherein R₁, R₁′ and R₁″ are each independently selected from the group consisting of H, alkyl, alkenyl, cycloalkyl, heterocycloalkyl, cycloalkenyl, heterocycloalkenyl, aryl, halogen, OH, an acyloxy group, and NR₈,R₉, wherein R₈ and R₉ are independently selected from the group consisting of H, alkyl, alkenyl, cycloalkyl, heterocycloalkyl, cycloalkenyl, heterocycloalkenyl, aryl, and acyl, any of which may be optionally substituted;
R₂, R₄, R₅ and R₇ are each independently H, alkyl, alkenyl, cycloalkyl, heterocycloalkyl, cycloalkenyl, heterocycloalkenyl, aryl, OH, acyloxy or halogen; and
R₃ and R₆ are each independently H, OH, acyloxy, NR₈R₉ or a halogen, wherein R₈ and R₉ are as defined above;
or an analog thereof; and pharmaceutically acceptable salts thereof.

In another embodiment of the invention, there are provided compounds having the structure of formula (Ia):

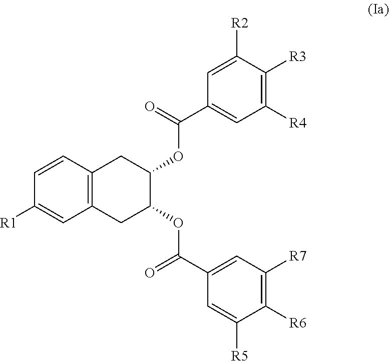

wherein R₁ is selected from the group consisting of H, alkyl, alkenyl, cycloalkyl, heterocycloalkyl, cycloalkenyl, heterocycloalkenyl, aryl, halogen, OH, an acyloxy group, and NR₈,R₉, wherein R₈ and R₉ are independently selected from the group consisting of H, alkyl, alkenyl, cycloalkyl, heterocycloalkyl, cycloalkenyl, heterocycloalkenyl, aryl, and acyl, any of which may be optionally substituted;
R₂, R₄, R₅ and R₇ are each independently H, alkyl, alkenyl, cycloalkyl, heterocycloalkyl, cycloalkenyl, heterocycloalkenyl, aryl, OH, acyloxy or halogen; and
R₃ and R₆ are each independently H, OH, acyloxy, NR₈R₉ or a halogen, wherein R₈ and R₉ are as defined above;
or an analog thereof; and pharmaceutically acceptable salts thereof.

In another embodiment of the invention there are provided compounds of formula (I) or (Ia), wherein R₁ is selected from the group consisting of H, halogen, OH, and an acyloxy group; R₂, R₄, R₅ and R₇ are each independently H, alkyl, alkenyl, cycloalkyl, heterocycloalkyl, cycloalkenyl, heterocycloalkenyl, aryl, OH, acyloxy or halogen; and R₃ and R₆ are each independently H, OH, acyloxy, NR₈R₉ or a halogen; and pharmaceutically acceptable salts thereof.

In accordance with another embodiment of the present invention, there is provided a compound having the structure of formula II:

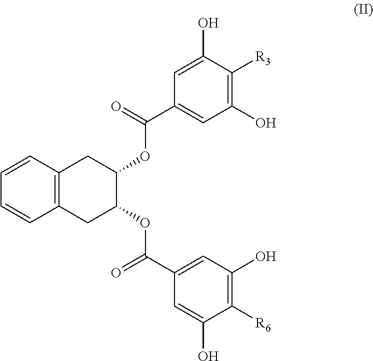

wherein:
R₃ and R₆ are both H, Br, F, Cl or CH₃;
or an analog thereof; and pharmaceutically acceptable salts thereof.

In accordance with yet another embodiment of the present invention, there is provided a compound having the structure of formula III:

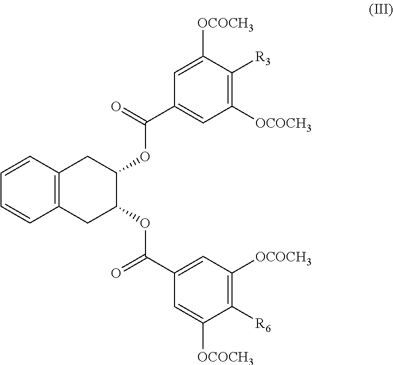

wherein:
R₃ and R₆ are both OCOCH₃, H, Br, F, Cl or CH₃;
or an analog thereof; and pharmaceutically acceptable salts thereof.

In accordance with yet another embodiment of the present invention, there is provided a compound having the structure of formula IV:

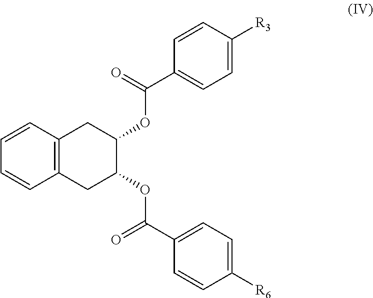

wherein:
R₃ and R₆ are both OH, OCOCH₃, NHCOOC(CH₃)₃, NH₂ or NHCOCH₃;
or an analog thereof;
and pharmaceutically acceptable salts thereof.

In another embodiment of the invention, there is provided a compound having the structure of formula V:

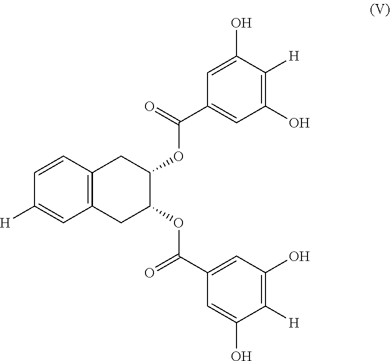

or an analog thereof; and pharmaceutically acceptable salts thereof.

In another embodiment of the invention, there is provided a compound having the structure of formula VI:

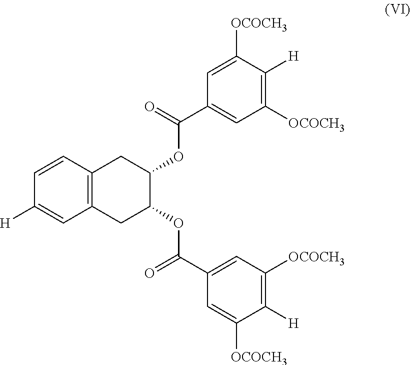

or an analog thereof; and pharmaceutically acceptable salts thereof.

In another embodiment of the invention, there is provided a compound having the structure of formula VII:

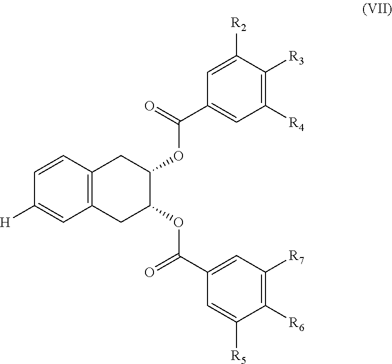

wherein R₂, R₃, R₄, R₅, R₆ and R₇ are F; or

### R₂, R₃, R₅ and R₆ are F, and R₄ and R₇ are H; or

### R₂, R₄, R₅ and R₇ are F, and R₃ and R₆ are H;

or an analog thereof;
and pharmaceutically acceptable salts thereof.

In another embodiment of the invention, there are provided compounds having the structure of formulae VIII, IX and X:

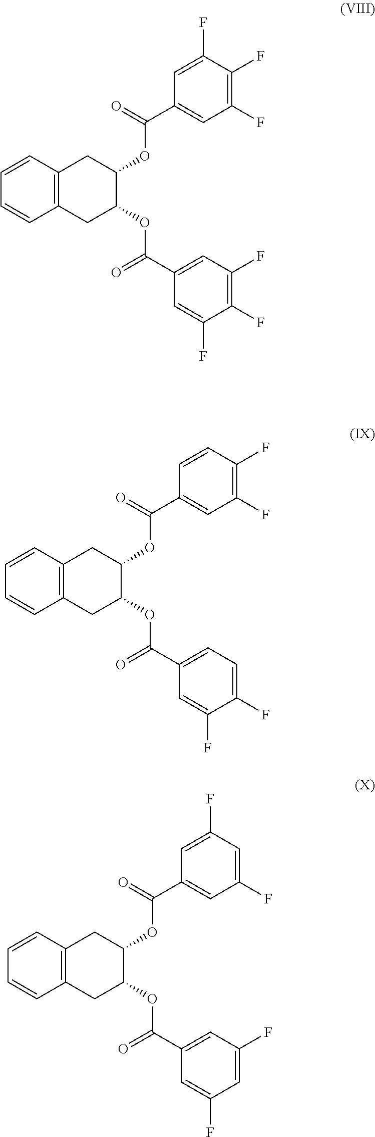

or an analog thereof; and pharmaceutically acceptable salts thereof.

In another embodiment, there are provided herein compounds having the structures described herein, for example the structures shown in Table 1, Scheme 1, Scheme 2, and Scheme 3, and analogs and pharmaceutically acceptable salts thereof. In one embodiment, the compound is an analog of a tea polyphenol. In an embodiment, the compound of the invention is an EGCG analog.

TABLE 1
Representative compounds of the invention.

| Compound # | Structure |
|---|---|
| 1 | 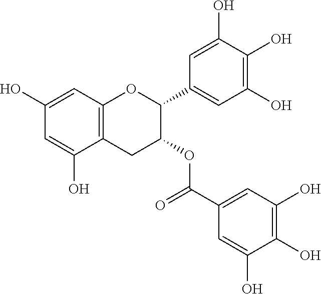 |
| 2 | 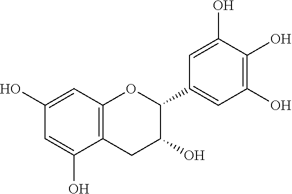 |
| 3 | 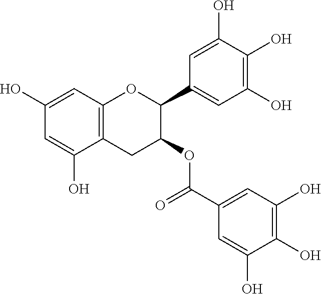 |
| 4 (Pro-EGCG) | 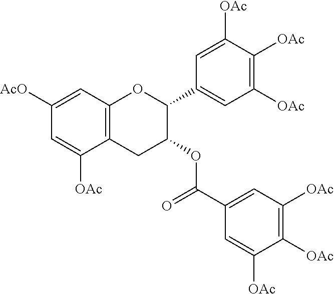 |
| 5: R₃ = OH, R₆ = OH 7: R₃ = H, R₆ = H 23: R₃ = Br, R₆ = Br 25: R₃ = CH₃, R₆ = CH₃ | 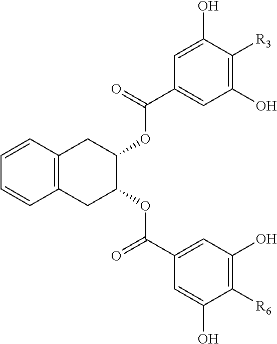 |
| 6: R₃ = OAc, R₆ = OAc 8: R₃ = H, R₆ = H 24: R₃ = Br, R₆ = Br 26: R₃ = CH₃, R₆ = CH₃ | 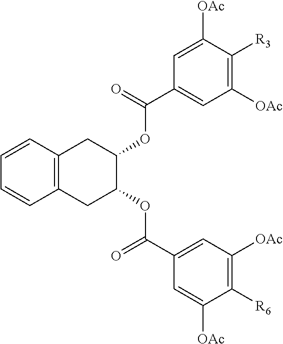 |
| 27: R₃ = OH, R₆ = OH 28: R₃ = Oac, R₆ = OAc 29: R₃ = NHBoc, R₆ = NHBoc 30: R₃ = NH₇ , R₆ = NH₂ 31: R₃ = NHAc, R₆ = NHAc | 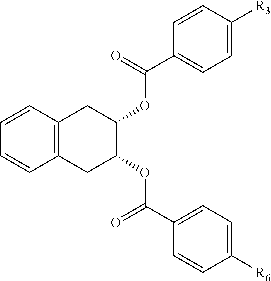 |
| 32 | 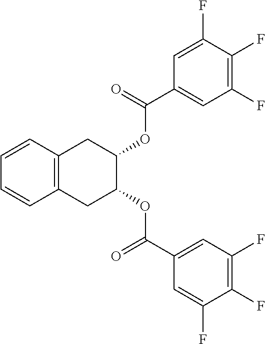 |
| 33 | 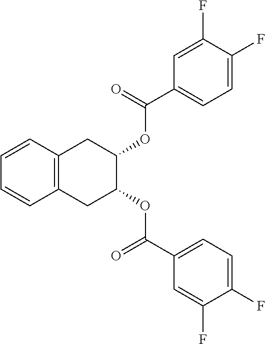 |
| 34 | 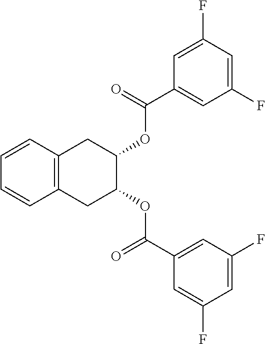 |
| 16 | 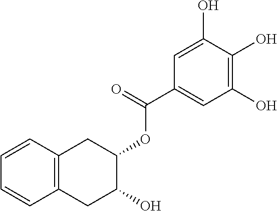 |
| 17 | 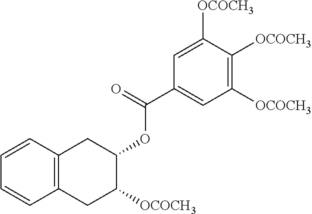 |
| 21 | 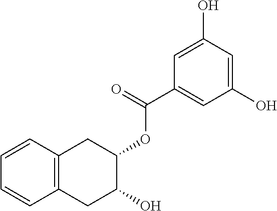 |
| 22 | 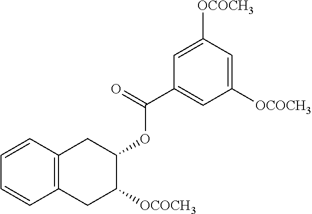 |

In an embodiment, there is provided herein a pharmaceutical composition comprising a compound of the invention (e.g. a compound of Formula I, Ia, II, III, IV, V, VI, VII, VIII, IX or X; a compound shown in Table 1; a compound shown in Scheme 1; a compound shown in Scheme 2; a compound shown in Scheme 3; or an EGCG analog) and one or more than one pharmaceutically acceptable carriers. Many pharmaceutically acceptable carriers are known in the art. It will be understood by those in the art that a pharmaceutically acceptable carrier must be compatible with the other ingredients of the formulation and tolerated by a subject in need thereof.

In another embodiment, the pharmaceutical composition comprises at least one additional active ingredient including, but are not limited to, antioxidants, free radical scavenging agents, peptides, growth factors, antibiotics, bacteriostatic agents, immunosuppressives, anticoagulants, buffering agents, anti-inflammatory agents, anti-pyretics, time-release binders, anesthetics, steroids and corticosteroids. In yet another embodiment, the pharmaceutical composition comprises at least one additional active ingredient including, but not limited to, other active ingredients commonly used in therapy for cancer such as Bortezomib (Velcade™), Taxol™, Docetaxel, Vinblastine, Vincristine, Camptothecin Toptecan, Etoposid, Teniposide and other natural, modified or synthetic chemotherapeutic agents known in the art. In a particular embodiment, the pharmaceutical composition of the invention comprises a compound of the invention and bortezomib (e.g. Velcade™) and a pharmaceutically acceptable carrier.

In an embodiment, the pharmaceutical compositions of the invention comprise a compound of formula (I) and a pharmaceutically acceptable carrier, optionally in association with at least one additional active agent. In another embodiment, the pharmaceutical compositions of the invention comprise a compound of formula (Ia), (II), (III), (IV), (V), (VI), (VII), (VIII), (IX) or (X); or a compound shown in Table 1; or a compound shown in Scheme 1, 2 or 3; and a pharmaceutically acceptable carrier, optionally in association with at least one additional active agent. In an embodiment, the at least one additional active agent is a therapeutic agent for cancer or a chemotherapeutic agent. In one embodiment, the at least one additional active agent is bortezomib.

In an aspect, the compounds and compositions provided herein may be used for inhibiting chymotrypsin-like activity of 20S proteasome and/or 26S proteasome, treating various types of cancer, and/or for inhibiting cancer cell growth.

In another aspect, the compounds and compositions of the invention comprise a compound selected from the group consisting of the compounds described herein, pharmaceutically acceptable salts, analogs, and mixtures thereof. The compound may be an analog of a tea polyphenol, e.g. an analog of EGCG. Pharmaceutically acceptable salts are known in the art and it should be understood that pharmaceutically acceptable salts of the compounds described herein are encompassed by the present invention.

Compositions and formulations of the invention include those suitable for oral, rectal, nasal, topical (including transdermal, buccal and sublingual), vaginal, parental (including subcutaneous, intramuscular, intravenous and intradermal) and pulmonary administration. Compositions of the present invention suitable for oral administration can be presented for example as discrete units such as capsules, cachets or tablets, each containing a predetermined amount of the active ingredient; or as an oil-in-water liquid emulsion, water-in-oil liquid emulsion or as a supplement within an aqueous solution. The active ingredient can also be presented as bolus, electuary, or paste. Formulations suitable for topical administration in the mouth include lozenges comprising the active ingredient, pastilles comprising the active ingredient in gelatin and glycerin, or sucrose and acacia.

Pharmaceutical compositions for topical administration according to the present invention can be formulated for example as an ointment, cream, suspension, lotion, powder, solution, paste, gel, spray, aerosol or oil. Alternatively, a formulation may comprise a patch or a dressing such as a bandage or adhesive plaster impregnated with active ingredients, and optionally one or more excipients or diluents.

Formulations suitable for topical administration to the eye also include eye drops wherein the active ingredient is dissolved or suspended in a suitable carrier, especially a sterile aqueous solvent for the agent. Formulations for rectal administration may be provided as a suppository with a suitable base comprising, for example, cocoa butter or a salicylate. Formulations suitable for vaginal administration can be presented as pessaries, tampons, creams, gels, pastes, foams or spray formulations containing in addition to the agent, such carriers as are known in the art to be appropriate.

Formulations suitable for nasal administration, wherein the carrier is a solid, include a coarse powder having a particle size, for example, in the range of about 20 to about 500 microns which is administered in the manner in which snuff is taken, i.e., by rapid inhalation through the nasal passage from a container of the powder held to the nose. Suitable formulations wherein the carrier is a liquid for administration by nebulizer, include for example aqueous or oily solutions of the agent.

Formulations suitable for parenteral administration include aqueous and non-aqueous isotonic sterile injection solutions which may contain preservatives, buffers, bacteriostatic agents and solutes which render the formulation isotonic with the blood of the patient; and aqueous and nonaqueous sterile suspensions which can include suspending agents and thickening agents, and liposomes or other microparticulate systems which are designed to target the compound to blood components or one or more organs. Extemporaneous injection solutions and suspensions can be prepared from sterile powders, granules and tablets.

It should be understood that in addition to the ingredients particularly mentioned above, the compositions and formulations of this invention can include other agents conventional in the art regarding the type of formulation in question. For example, formulations suitable for oral administration can include such further agents as sweeteners, thickeners, and flavoring agents. It also is intended that the agents, compositions, and methods of this invention be combined with other suitable compositions and therapies.

Various delivery systems are known and can be used to administer a therapeutic agent of the invention, e.g., encapsulation in liposomes, microparticles, microcapsules and the like. Methods of delivery include, but are not limited to, intraarterial, intramuscular, intravenous, intranasal, and oral routes. In a specific embodiment, the compounds and pharmaceutical compositions of the invention can be administered locally to the area in need of treatment; such local administration can be achieved, for example, by local infusion during surgery, by injection, or by means of a catheter.

Therapeutic amounts can be empirically determined and will vary with the pathology being treated, body mass of the subject being treated, and the efficacy and toxicity of the agent. Similarly, suitable dosage formulations and methods of administering the agents can be readily determined by those of skill in the art. For example, a daily dosage can be divided into one, two or more doses in a suitable form to be administered at one, two or more times throughout a time period.

The compounds and pharmaceutical compositions can be administered by any of a variety of routes, such as orally, intranasally, parenterally or by inhalation, and can take the form, for example, of tablets, lozenges, granules, capsules, pills, ampoule, suppositories or aerosol form. They can also be in the form of suspensions, solutions, and emulsions of the active ingredient in aqueous or nonaqueous diluents, syrups, granulates or powders. In addition to an agent of the present invention, the pharmaceutical compositions can also contain other pharmaceutically active compounds.

Pharmaceutical compositions of the present invention suitable for oral administration may be presented as discrete units such as capsules, cachets or tablets each containing a predetermined amount of the active ingredient, as a powder or granules or as a solution or a suspension in an aqueous liquid, a non-aqueous liquid, an oil-in-water emulsion or a water-in-oil liquid emulsion. Such compositions may be prepared by any of the methods of pharmacy but all methods include the step of bringing into association the active ingredient with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. For example, in an embodiment each tablet may contain from about 2.5 mg to about 500 mg of the active ingredient and each cachet or capsule may contain from about 2.5 to about 500 mg of the active ingredient.

The magnitude of prophylactic or therapeutic dose of a compound of the invention will, of course, vary with the nature of the severity of the condition to be treated and with the particular compound of the invention and its route of administration. It will also vary according to the age, weight and response of the individual patient. In general, the daily dose range for treating cancer lies within the range of from about 0.001 mg to about 100 mg per kg body weight of a mammal, preferably 0.01 mg to about 10 mg per kg, and most preferably 0.1 to 1 mg per kg, in single or divided doses. On the other hand, it may be necessary to use dosages outside these limits in some cases.

For use where a composition for intravenous administration is employed, in an embodiment a suitable dosage range for treating cancer is from about 0.001 mg to about 25 mg (preferably from 0.01 mg to about 1 mg) of a compound of the invention per kg of body weight per day.

In the case where an oral composition is employed, in an embodiment a suitable dosage range for treating cancer is, e.g. from about 0.01 mg to about 100 mg of a compound of the invention per kg of body weight per day, preferably from about 0.1 mg to about 10 mg per kg.

Ideally, the therapeutic agent of the invention should be administered to achieve peak concentrations of the active compound at sites of the disease. Peak concentrations at disease sites can be achieved, for example, by intravenously injecting the agent, optionally in saline, or orally administering, for example, a tablet, capsule or syrup containing the active ingredient.

Advantageously, the compounds and compositions of the invention can be administered simultaneously or sequentially with other drugs or biologically active agents. Examples include, but are not limited to, antioxidants, free radical scavenging agents, peptides, growth factors, antibiotics, bacteriostatic agents, immunosuppressives, anticoagulants, buffering agents, anti-inflammatory agents, anti-pyretics, time-release binders, anesthetics, steroids and corticosteroids; other anti-cancer therapeutics and chemotherapeutic agents such as bortezomib (Velcade™), Taxol™, docetaxel, vinblastine, vincristine, camptothecin toptecan, etoposid, and teniposide; and other proteasome inhibitors such as bortezomib (Velcade™), salinosporamide, and epigallocatechin gallate and its analogs.

Accordingly, in the methods and uses of the present invention the compounds of the invention can also be administered in combination with other therapeutic agents. In an embodiment, the present invention provides a method of treating cancer, e.g. multiple myeloma, comprising administering to a subject in need thereof an effective amount of a first agent comprising a compound or composition of the invention, and a second agent. The second agent may be, for example, an anti-cancer therapeutic or chemotherapeutic agent, e.g. bortezomib.

Administration in combination with another agent includes co-administration (simultaneous administration of a first and second agent) and sequential administration (administration of a first agent, followed by the second agent, or administration of the second agent, followed by the first agent). The combination of agents used within the methods described herein may have a therapeutic additive or synergistic effect on the condition(s) or disease(s) targeted for treatment. The combination of agents used within the methods described herein also may reduce a detrimental effect associated with at least one of the agents when administered alone or without the other agent(s). For example, the toxicity of side effects of one agent may be attenuated by the other, thus allowing a higher dosage, improving patient compliance, or improving therapeutic outcome. Physicians may achieve the clinical benefits of previously recognized drugs while using lower dosage levels, thus minimizing adverse side effects. In addition, two agents administered simultaneously and acting on different targets may act synergistically to modify or ameliorate disease progression or symptoms.

Another aspect of the present invention is directed to methods of inhibiting proteasomal activity. In particular, without limitation the chymotrypsin activity and/or chymotrypsin-like activity of the 20S proteasome may be inhibited.

In an aspect, a method of inhibiting proteasomal activity is provided, comprising contacting a cell with a sufficient amount of a compound or composition of the invention.

In another aspect, the present invention provides a method of inhibiting chymotrypsin-like activity of the 20S proteasome, comprising administering to a subject a therapeutically effective amount of a compound or pharmaceutical composition of the present invention.

In accordance with another embodiment of the present invention, there is provided a method of treating cancer, comprising administering to a subject a therapeutically effective amount of a compound or pharmaceutical composition of the present invention.

A cancer to be treated in accordance with an embodiment of the present invention may be selected from the group consisting of, but not limited to, prostate cancer, leukemia, hormone dependent cancers, breast cancer, colon cancer, lung cancer, epidermal cancer, liver cancer, esophageal cancer, stomach cancer, cancer of the brain, and cancer of the kidney. In one embodiment, the cancer is multiple myeloma.

In another embodiment of the present invention, there is provided herein a method of inhibiting tumor cell growth, comprising administering to a subject a therapeutically effective amount of a compound or pharmaceutical composition of the present invention.

For the purpose of the present invention the following terms are defined below:

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at east a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “inhibition” is intended to mean a substantial slowing, interference, suppression, prevention, delay and/or arrest of a chemical or biochemical action.

The term “pharmacological inhibition” is intended to mean a substantial slowing, interference, suppression, prevention, delay and/or arrest of a chemical action which is caused by an effective amount of a compound, drug, or agent.

The term “inhibitor” is intended to mean a compound, drug, or agent that substantially slows, interferes, suppresses, prevents, delays and/or arrests a chemical action.

The term “polyphenol” is intended to mean a compound with more than one phenolic moiety. A phenolic compound is an aromatic compound containing an aromatic nucleus to which is directly bonded at least one hydroxyl group. The term polyphenol includes, without limitation, (−)EGCG, (−)EGC, (−)ECG, and (−)EC, such as those that can be extracted from leaves of the tea plant Camellia sinensis, and analogs thereof, as well as structurally similar synthetic analogs.

The term “per-acetate” or “per-acetylated” or “per-acylated”, as used herein is intended to mean a polyphenol that is connected by a group such that all the hydroxyl groups of the polyphenol are acylated.

The term “alkyl group”, as used herein, is understood as referring to a saturated, monovalent unbranched or branched hydrocarbon chain. Examples of alkyl groups include, but are not limited to, C₁₋₁₀ alkyl groups. Examples of C₁₋₁₀ alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, nonyl and decyl.

The term “aryl”, as used herein, is understood as referring to 5-, 6- and 7- or more membered aromatic groups, for example phenyl or naphthyl, that may include from zero to four heteroatoms selected independently from O, N and S in the ring, for example, pyrrolyl, furyl, thiophenyl, imidazolyl, oxazole, thiazolyl, triazolyl, pyrazolyl, pyridyl, pyrazinyl, pyridazinyl and pyrimidinyl, and the like. Those aryl groups having heteroatoms in the ring structure may also be referred to as “aryl heterocycles” or “heteroaryl”. The aromatic ring can be substituted at one or more ring positions. Aryl groups can also be part of a polycyclic group. For example, aryl groups include fused aromatic moieties such as naphthyl, anthracenyl, quinolyl, indolyl, and the like.

The term “acyl group” is intended to mean a group having the formula RC=O, wherein R is an alkyl, alkenyl, cycloalkyl, heterocycloalkyl, cycloalkenyl, heterocycloalkenyl, or an aryl group.

The term “alkenyl” refers to a straight or branched chain alkyl moiety having two or more carbon atoms (e.g., two to six carbon atoms, C₂₋₆ alkenyl) and having in addition one double bond, of either E or Z stereochemistry where applicable. This term would include, for example, vinyl, 1-propenyl, 1- and 2-butenyl, 2-methyl-2-propenyl, etc.

The term “cycloalkyl” refers to a saturated alicyclic moiety having three or more carbon atoms (e.g., from three to six carbon atoms) and which may be optionally benzofused at any available position. This term includes, for example, cyclopropyl, cyclobutyl, cyclopentyl, cyclohexyl, indanyl and tetrahydronaphthyl.

The term “heterocycloalkyl” refers to a saturated heterocyclic moiety having three or more carbon atoms (e.g., from three to six carbon atoms) and one or more heteroatom from the group N, O, S (or oxidised versions thereof) and which may be optionally benzofused at any available position. This term includes, for example, azetidinyl, pyrrolidinyl, tetrahydrofuranyl, piperidinyl, indolinyl and tetrahydroquinolinyl.

The term “cycloalkenyl” refers to an alicyclic moiety having three or more carbon atoms (e.g., from three to six carbon atoms) and having in addition one double bond. This term includes, for example, cyclopentenyl or cyclohexenyl.

The term “heterocycloalkenyl” refers to an alicyclic moiety having from three to six carbon atoms and one or more heteroatoms from the group N, O, S (or oxides thereof) and having in addition one double bond. This term includes, for example, dihydropyranyl.

The term “halogen” means a halogen atom such as fluorine, chlorine, bromine, or iodine.

The term “optionally substituted” means optionally substituted with one or more of the aforementioned groups (e.g., alkyl, aryl, heteroaryl, acyl, alkenyl, cycloalkyl, heterocycloalkyl, cycloalkenyl, heterocycloalkenyl, or halogen), at any available position or positions.

The term “analog” is intended to mean a compound that is similar or comparable, but not identical, to a reference compound, i.e. a compound similar in function, structure, properties and/or appearance to the reference compound. For example, the reference compound can be a reference green tea polyphenol and an analog is a substance possessing a chemical structure or chemical properties similar to those of the reference green tea polyphenol. As used herein, an analog is a chemical compound that may be structurally related to another but differs in composition (for example as in the replacement of one atom by an atom of a different element or in the presence of a particular functional group). An analog may be derived from a natural source or be prepared using chemical synthesis.

The term “cancer” is intended to mean any cellular malignancy whose unique trait is the loss of normal controls which results in unregulated growth, lack of differentiation and ability to invade local tissues and metastasize. More specifically, cancer is intended to include, without limitation, prostate cancer, leukemia, hormone dependent cancers, breast cancer, colon cancer, lung cancer, epidermal cancer, liver cancer, esophageal cancer, stomach cancer, cancer of the brain, cancer of the kidney and multiple myeloma.

The terms “treatment” or “treating” are intended to mean obtaining a desired pharmacologic and/or physiologic effect, such as inhibition of cancer cell growth or induction of apoptosis of a cancer cell or an improvement in a disease condition in a subject or improvement of a symptom associated with a disease or a medical condition in a subject. The effect may be prophylactic in terms of completely or partially preventing a disease or symptom associated therewith and/or may be therapeutic in tetras of a partial or complete cure for a disease and/or the pathophysiologic effect attributable to the disease. “Treatment” as used herein covers any treatment of a disease in a mammal and includes: (a) preventing a disease or condition (such as preventing cancer) from occurring in an individual who may be predisposed to the disease but has not yet been diagnosed as having it; (b) inhibiting the disease, (e.g., arresting its development); or (c) relieving the disease (e.g., reducing symptoms associated with the disease).

The term “biological activity” is intended to mean having the ability to inhibit the proteasome, e.g. to inhibit the chymotrypsin-like activity of the proteasome, having the ability to inhibit cell growth, induce apoptosis, and/or suppress the transforming activity in cancer cells. “Biological activity” also means having therapeutic efficacy and/or the ability to treat cancer in a subject.

The term “therapeutically effective” is intended to mean an amount of a compound sufficient to substantially improve a symptom associated with a disease or a medical condition or to improve, ameliorate or reduce the underlying disease or medical condition. For example, in the treatment of cancer, a compound which decreases, prevents, delays, suppresses, or arrests any symptom of the disease would be therapeutically effective. A therapeutically effective amount of a compound may provide a treatment for a disease such that the onset of the disease is delayed, hindered, or prevented, or the disease symptoms are ameliorated, or the term of the disease is altered.

The term “chymotrypsin-like activity” refers to the ability of the eukaryotic proteasome subunit to cleave amino acid sequences after hydrophobic residues, and is intended to include chymotrypsin activity.

As used herein, the term “subject” includes mammals, including humans.

When the compounds of this invention are administered in combination with other agents, they may be administered sequentially or concurrently to an individual. Alternatively, pharmaceutical compositions according to the present invention may be comprised of a combination of analogs of the present invention, as described herein, and another therapeutic or prophylactic agent known in the art.

It will be understood that a specific “effective amount” for any particular in vivo or in vitro application will depend upon a variety of factors including the activity of the specific compound employed, the age, body weight, general health, sex, and/or diet of the individual, time of administration, route of administration, rate of excretion, drug combination and the severity of the particular disease being treated. For example, the “effective amount” may be the amount of polyphenol compound of the invention necessary to achieve inhibition of proteosomal chymotrypsin-like activity in vivo or in vitro.

The terms “UPS” and “the proteasome” are used interchangeably throughout the specification.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include citric acid, lactic acid, tartaric acid, fatty acids, and the like. Pharmaceutically acceptable salts are known in the art.

Salts may also be formed with bases. Such salts include salts derived from inorganic or organic bases, for example alkali metal salts such as magnesium or calcium salts, and organic amine salts such as morpholine, piperidine, dimethylamine or diethylamine salts.

As used herein, the term “pharmaceutically acceptable carrier” includes any and all solvents such as phosphate buffered saline, water, saline, dispersion media, coatings, antibacterial and antifungal agents, isotonic and absorption delaying agents and the like. The use of such media and agents for pharmaceutically active substances is well known in the art. Supplementary active ingredients can also be incorporated into the compositions. The pharmaceutical compositions of the invention can be formulated according to known methods for preparing pharmaceutically useful compositions. Formulations are described in a number of sources which are well known and readily available to those skilled in the art. For example, Remington's Pharmaceutical Science (Martin E W (1995) Easton Pa., Mack Publishing Company, 19th ed.) describes formulations which can be used in connection with the subject invention.

The present description refers to a number of chemical terms and abbreviations used by those skilled in the art. Nevertheless, definitions of selected terms are provided for clarity and consistency.

Abbreviations: OsO4: Osmium tetroxide; NMO: N-Methylmorpholine-N-Oxide; Ac₂O: Acetic anhydride; Py: Pyridine; TFA: Trifluoroacetic acid; HBTU: O-(Benzotriazol-1-yl)-N,N,N′,N′-tetramethyluronium hexa-fluorophosphate; HOBT: 1-Hydroxybenzotriazole; DIPEA: N,N-Diisopropylethylamine; DMAP: 4-Dimethylaminopyridine; DCC: 1,3-Dicyclohexylcarbodiimide; Bn: Benzyloxy; MeOH: Methanol; TLC: Thin Layer Chromatography; NMR: Nuclear Magnetic Resonance; MS: Mass Spectroscopy; ESI: Electrospray Ionization; FAB: Fast Atom Bombardment; PEG: Polyethylene Glycol; SPE: Solid-Phase Extraction; IL: TGA: Thermogravimetric Analysis; RP: Residue Percentage; ODT: Onset Decomposing Temperatures; DMF: Dimethylformamide; DMSO: Dimethyl Sulfoxide; THF: Tetrahydrofuran; DNC: 3,5-dinitrocatechol; (−)-EGCG: (−)Epigallocatechin gallate; Pro-EGCG: (−)Epigallocatechin gallate octa acetate.

### EXAMPLES

The present invention will be more readily understood by referring to the following examples, which are provided to illustrate the invention and are not to be construed as limiting the scope thereof in any manner.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of the invention.

### Methods of Synthesis

Compounds of the invention can be prepared according to the synthetic routes outlined below and by following the methods described herein.

The preparation of the compounds of the present invention is illustrated in Schemes 1, 2 and 3. With reference to compounds 5 and 7 and their respective per-acetates 6 and 8, 1,4-Dihydronaphthalene 11 was dihydroxylated with osmium tetraoxide affording the cis-diol 12. Compound 12 is treated with DCC/DMAP and one molar equivalent of benzyl-protected gallic acid affording the corresponding monobenzoate 14. When more than two equivalents of benzyl-protected gallic acid is used in the reaction sequence, the dibenzoate 15 is obtained. Removal of the O-benzyl protecting group of 14 and 15 by palladium catalyzed hydrogenolysis gave compounds 16 and 5 respectively. Acetylation of 16 and 5 gave the corresponding acetates 17 and 6. A similar reaction sequence of 12 with 3,5-dibenzyloxybenzoic acid gave the series 21 and 7 which were converted to their respective acetates 22 and 8.

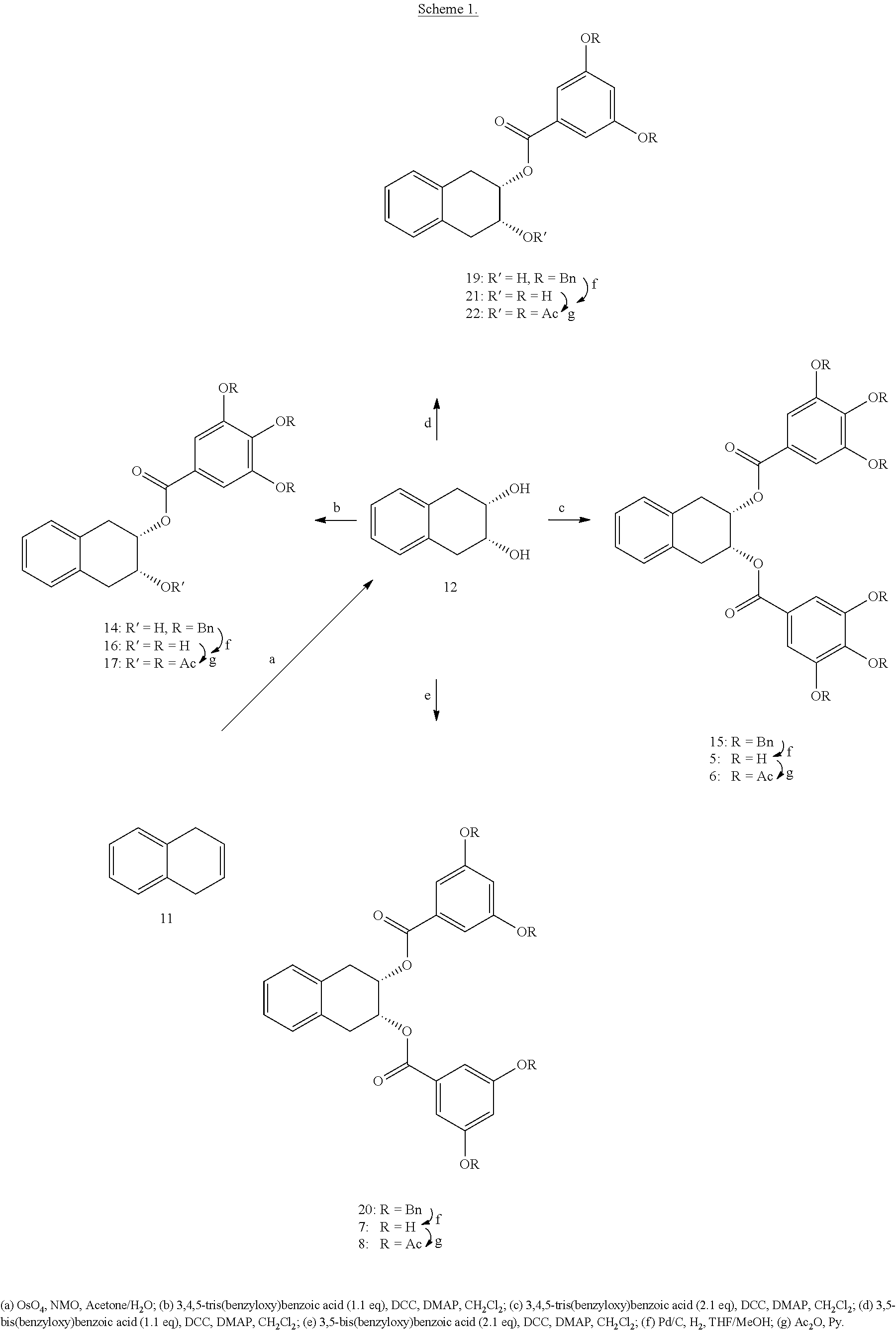

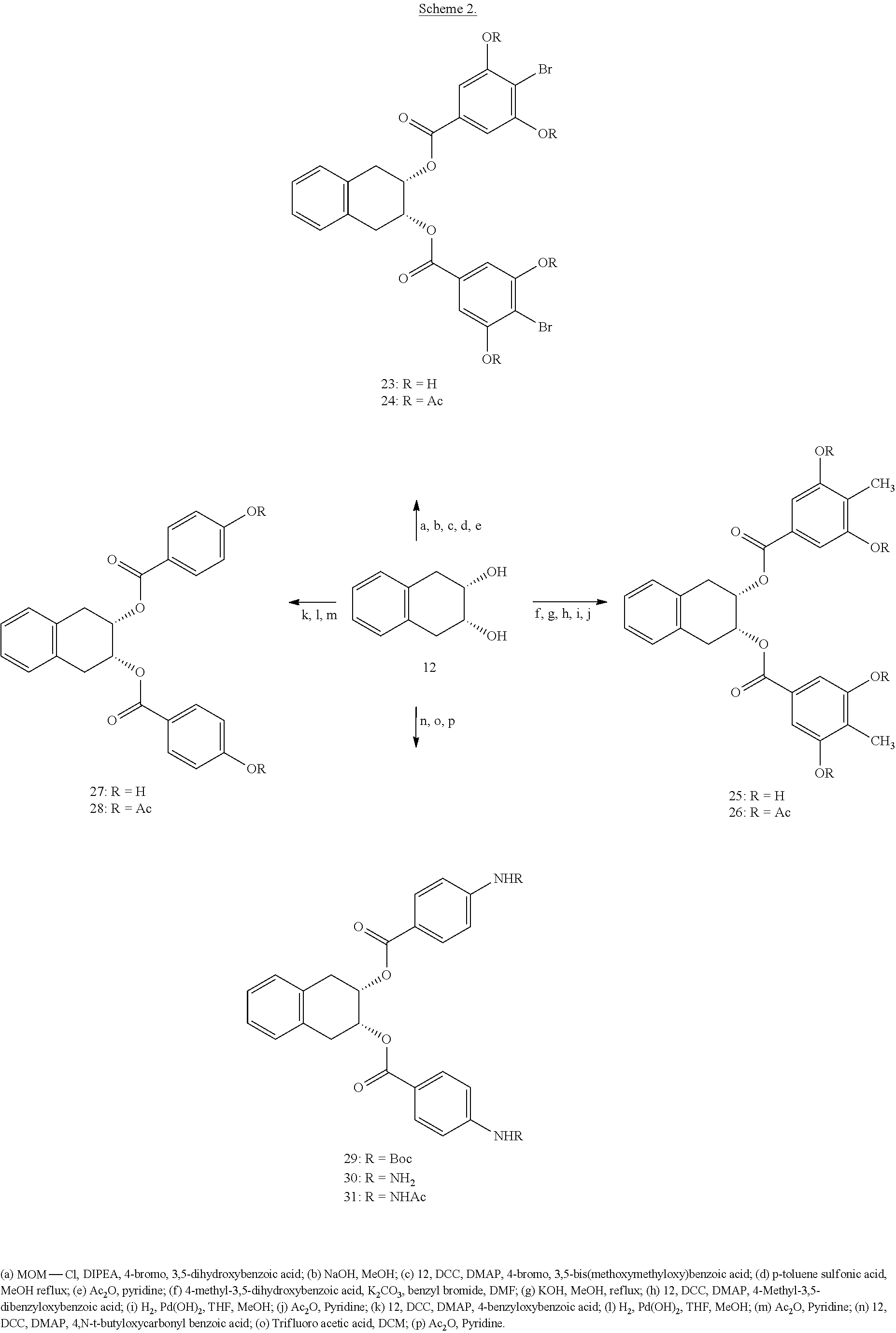

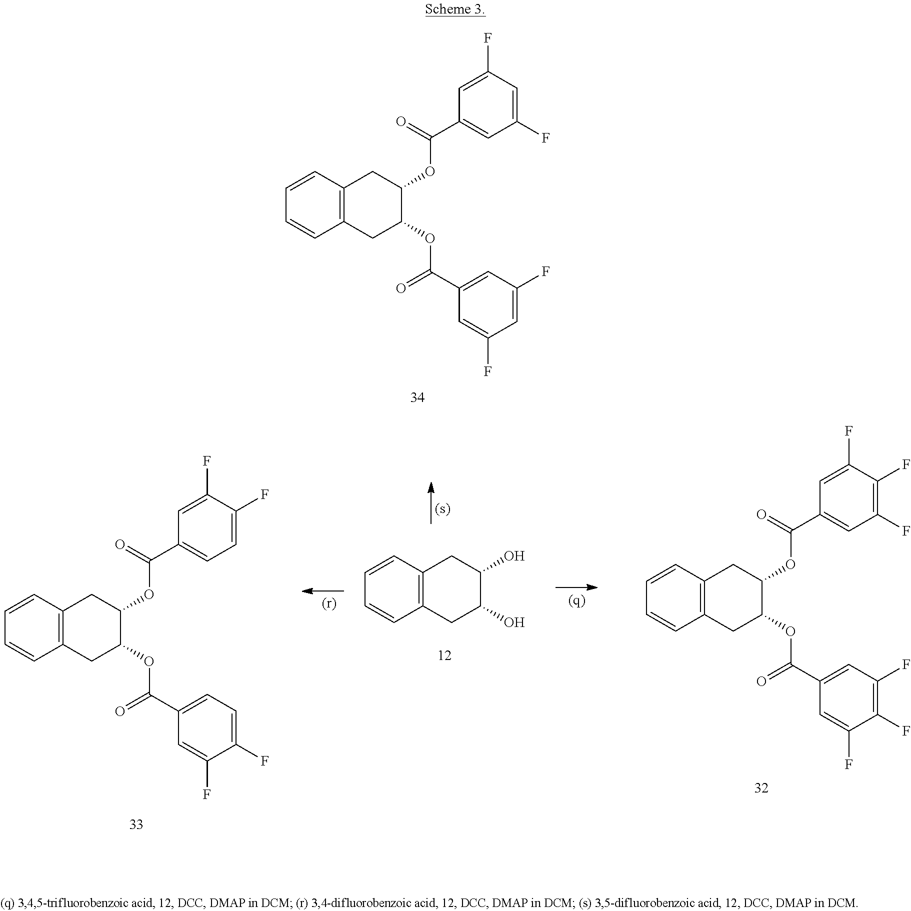

### Experimental Methods

General methods. The starting materials and reagents, purchased from commercial suppliers, were used without further purification. Anhydrous methylene chloride was distilled under nitrogen from CaH₂. Anhydrous DMF was distilled under vacuum from CaH₂. Reaction flasks were flame-dried under a stream of N₂. All moisture-sensitive reactions were conducted under a nitrogen atmosphere. Flash chromatography was carried out using silica-gel 60 (70-230 mesh). The melting points were uncorrected. ¹H-NMR and ¹³C NMR (300 MHz) spectra were measured with TMS as an internal standard when CDCl₃, CD₃OD and acetone-d₆ were used as solvent. High-resolution (ESI) MS spectra were recorded using a QTOF-2 Micromass spectrometer.

### Biological Assays

Materials. Purified rabbit 20S proteasome and fluorogenic substrate Suc-LLVY-AMC for the proteasomal chymotrypsinlike (CT-like) activity were obtained from Calbiochem Inc. (San Diego, Calif.). Fetal bovine serum (FBS) was from Tissue Culture Biologicals (Tulare, Calif.). Penicillin and streptomycin were purchased from Invitrogen Co. (Carlsbad, Calif.). RPMI 1640 medium was purchased from Invitrogen Co. (Carlsbad, Calif.). MTT (3-4,5-dimethylthiazol-2-yl-2,5-diphenyl-tetrazolium bromide) was purchased from Sigma-Aldrich.

Cell culture. Human breast cancer MDA-MB-231 cells were purchased from American Type Culture Collection (Manassas, Va.) and grown in RPMI 1640 medium supplemented with 10% FBS, 100 units/ml of penicillin, and 100 μg/ml of streptomycin. Cells were maintained at 37° C. and 5% CO₂.

### Example 1

### Preparation of cis-1,2,3,4-tetrahydro-naphthalene-2,3-diol (12)

To a solution of 1,4-dihydronaphthalene (500 mg, 3.84 mmol) in acetone/H₂O (3.0/1.0 mL) was added a solution of NMO in H₂O (1.43 mL, 50% wt, 6.90 mmol) and a solution of OsO₄ in 2-methyl-2-propanol (313 μL, 2.5% wt., 25 μmol). The mixture was stirred at room temperature for 16 h. Saturated Na₂SO₃ aqueous solution (10 mL) was added and stirred for an additional 15 min. H₂O (10 mL) and EtOAc (30 mL) was added and stirred for 5 min. The aqueous phase was extracted with EtOAc (4×30 mL). The combined organic phase was washed with brine (20 mL) and dried over anhydrous Na₂SO₄. The solution was concentrated by rotary evaporator and vacuum drying to give the crude product which was purified by silica gel chromatography (Hexane/EtOAc/CH₂Cl₂=5/1/1) to afford 521.7 mg (83%) of the title compound as a white solid. ¹H NMR (acetone-d₆, 300 MHz) δ 7.13 (m, 2H), 7.09 (m, 2H), 4.11 (t, J=5.4, 2 H), 3.01 (m, 4H), 2.36 (s, 2H); ¹³C NMR (acetone-d₆, 75 MHz) δ 132.87, 129.11, 126.25, 69.24, 34.32.

### Preparation of Monobenzoates 14 and 19

To a solution of the corresponding benzoic acid (0.22 mmol) in dry CH₂Cl₂ (20 mL), dicyclohexylcarbodiimide (DCC, 45 mg, 0.22 mmol) was added. The resulting mixture was stirred at room temperature for 4 h, 4-Dimethylaminopyridine (DMAP, 3 mg, 0.025 mmol.) was added, then a solution of diol 12 (33 mg, 0.2 mol) in CH₂Cl₂ (5 mL) was added dropwise. The mixture was stirred at room temperature overnight. Then the mixture was concentrated, Ethyl acetate (1 mL) was added and cooled in fridge, the urea byproduct was filtered and the filtrate was evaporated. The resulting residue was purified by column chromatography (Ethyl acetetate/n-Hexane=1:3) to afford the desired compound as a pale yellow amorphous solid.

### Compound 14:

white solid (61% yield). ¹H NMR (CDCl₃, 300 MHz) δ 7.43-7.13 (m, 22H), 5.48 (brs, 1H), 5.15 (s, 2H), 5.11 (s, 4H), 4.33 (s, 1H), 3.28-3.03 (m, 4H); ¹³C NMR (CDCl₃, 75 MHz) δ 166.4, 152.7, 142.7, 137.6, 136.9, 133.3, 132.7, 129.5, 129.3, 128.9, 128.8, 128.5, 128.3, 127.8, 126.7, 126.6, 125.3, 109.4, 75.4, 73.4, 71.4, 69.8, 68.1, 35.0, 32.2.

### Compound 19

white solid (65% yield). ¹H NMR (CDCl₃, 300 MHz) δ 7.44-7.13 (m, 16H), 6.82 (s, 1H), 5.50 (brs, 1H), 5.14 (s, 1H), 5.05 (s, 4H), 4.35 (s, 1H), 3.31-3.08 (m, 4H); ¹³C NMR (CDCl₃, 75 MHz) δ 166.6, 160.0, 136.8, 133.4, 132.8, 132.3, 129.6, 129.3, 129.0, 128.5, 128.0, 126.7, 126.7, 108.9, 107.4, 73.7, 70.6, 68.0, 35.0, 32.1.

### Preparation of Dibenzoates 15 and 20

To a solution of 12 (33 mg, 0.2 mmol) in CH₂Cl₂ (3.0 mL) were added the corresponding benzoic acid (0.42 mmol), 4-Dimethylaminopyridine (DMAP, 6 mg, 0.05 mmol) and dicyclohexylcarbodiimide (DCC, 87 mg, 0.42 mmol). The mixture was stirred at room temperature overnight. Then the mixture was concentrated, EA (1 mL) was added and cooled in fridge, the urea byproduct was filtered and the filtrate was evaporated. The resulting residue was purified by column chromatography (EA/n-Hex=1:6) to afford the desired compound as white amorphous solid.

### Compound 15

white solid (59% yield). ¹H NMR (CDCl₃, 300 MHz) δ 7.43-7.22 (m, 38H), 5.72 (brs, 1H), 5.01-4.96 (m, 12H), 3.38 (dd, J=17.4 Hz, J=4.8 Hz, 1H), 3.25 (dd, J=17.4 Hz, J=6.9 Hz, 1H); ¹³C NMR (CDCl₃, 75 MHz) δ 165.7, 152.7, 142.7, 137.7, 136.7, 132.6, 129.4, 128.8, 128.7, 128.5, 128.3, 128.2, 127.8, 126.9, 125.2, 109.2, 75.3, 71.2, 70.5, 32.5.

### Compound 20

white solid (71% yield). ¹H NMR (CDCl₃, 300 MHz) δ 7.42-7.18 (m, 28H), 6.74 (s, 1H), 5.72 (brs, 1H), 4.91 (m, 9H), 3.34 (m, 4H); ¹³C NMR (CDCl₃, 75 MHz) δ 165.9, 160.0, 136.6, 132.5, 132.2, 129.4, 128.8, 128.4, 127.9, 126.8, 108.6, 107.8, 70.6, 70.4, 32.3.

### General Procedures for Palladium Catalyzed Hydrogenolysis: Preparation of 5, 7, 16 and 21

To a solution of benzylated substrate (0.1 mmol) in THF/MeOH (3 mL, 1:2) was added palladium hydroxide (20 mg, 20% on carbon). The resulting mixture was stirred at room temperature until TLC showed that the reaction was completed (within 6 h). Then the reaction mixture was filtered to remove the catalyst. The filtrate was evaporated to afford product as a white solid.

### Compound 16

white solid (95% yield). ¹H NMR (CD₃OD, 300 MHz) δ 7.13-7.01 (m, 6H), 5.39 (m, 1H), 4.25 (m, 1H), 3.14-3.06 (m, 4H); ¹³C NMR (CD₃OD, 75 MHz) δ 167.1, 145.2, 138.6, 133.5, 132.8, 129.0, 128.8, 126.1, 126.0, 120.6, 109.0, 72.5, 67.4, 34.4, 32.1; HRMS m/z calculated for C₁₇H₁₆O₆Na 339.0840, found 339.0839.

### Compound 5

white solid (95% yield). ¹H NMR (CD₃OD, 300 MHz) δ 7.21-7.16 (m, 4H), 7.09 (s, 4H), 5.61 (m, 2H), 3.37-3.25 (m, 4H); ¹³C NMR (CD₃OD, 75 MHz) δ 165.3, 145.1, 138.0, 132.8, 129.0, 126.3, 120.9, 109.0, 69.7, 31.9; HRMS m/z calculated for C₂₄H₂₀O₁₀Na 491.0947, found 491.0949.

### Compound 21

white solid (95% yield). NMR (CD₃OD, 300 MHz) δ 7.14-7.07 (m, 4H), 6.92 (s, 2H), 6.44 (s, 1H), 5.43 (m, 1H), 4.27 (m, 1H), 3.17-3.10 (m, 4H); ¹³C NMR (CD₃OD, 75 MHz) δ 170.6, 166.0, 134.2, 133.2, 132.9, 129.3, 129.1, 126.3, 126.2, 108.2, 107.4, 73.2, 67.2, 35.0, 32.1; HRMS m/z calculated for C₁₇H₁₆O₅Na 323.0891, found 323.0890.

### Compound 7

white solid (95% yield). ¹H NMR (CD₃OD, 300 MHz) δ 7.16 (s, 4H), 6.88 (s, 4H), 6.46 (s, 2H), 5.66 (m, 2H), 3.31 (m, 4H); ¹³C NMR (CD₃OD, 75 MHz) δ 166.3, 158.6, 132.5, 131.9, 128.9, 126.4, 107.7, 107.3, 70.4, 31.8; HRMS m/z calculated for C₂₄H₂₀O₈ Na 459.1047, found 459.1050.

### Preparation of the Acetates 6, 8, 17 and 22

To a solution of the corresponding substrate (0.1 mmol) and acetic anhydride (0.5 mL) in pyridine (0.5 mL) at room temperature. The resulting mixture was stirred overnight. Then EA (50 mL) was added and 1N HCl (1 mL) and washed with CuSO4 solution (3×10 mL), water (2×10 mL) and brine (10 mL), dried over sodium sulfate and evaporated. The residue was purified by column chromatography over silica gel (Ethyl acetate/n-Hexane 3:2) to afford the title product as white solid.

### Compound 17

white solid (92% yield). ¹H NMR (CDCl₃, 300 MHz) δ 7.74 (s, 2H), 7.21-7.11 (m, 4H), 5.64 (m, 1H), 5.42 (m, 1H), 3.26-3.16 (m, 4H), 2.30 (s, 9H), 2.07 (s, 3H); ¹³C NMR (CDCl₃, 75 MHz) δ 170.9, 167.8, 166.6, 164.1, 143.7, 139.0, 132.6, 132.3, 129.3, 128.6, 126.8, 126.7, 122.5, 70.9, 69.7, 32.5, 31.7, 21.4, 20.8, 20.4; HRMS m/z calculated for C₂₅H₂₄O₁₀Na 507.1266, found 507.1262.

### Compound 6

white solid (94% yield). ¹H NMR (CDCl₃, 300 MHz) δ 7.73-7.70 (m, 4H), 7.22-7.14 (m, 4H), 5.68 (m, 2H), 3.31 (m, 4H), 2.28 (s, 18H); ¹³C NMR (CDCl₃, 75 MHz) δ 167.9, 166.6, 164.1, 143.7, 139.1, 132.2, 129.4, 128.4, 126.9, 122.6, 71.1, 32.1, 20.8, 20.4; HRMS m/z calculated for C₃₆H₃₂O₁₆Na 743.1576, found 743.1583.

### Compound 22

white solid (90% yield). ¹H NMR (CDCl₃, 300 MHz) δ 7.60 (s, 2H), 7.21-7.11 (m, 5H), 5.64 (m, 1H), 5.44 (m, 1H), 3.26-3.17 (m, 4H), 2.31 (s, 6H), 2.08 (s, 3H); ¹³C NMR (CDCl₃, 75 MHz) δ 170.9, 169.0, 164.6, 151.1, 132.6, 132.5, 132.3, 129.3, 126.8, 126.7, 120.7, 120.6, 70.8, 69.7, 32.4, 31.8, 21.4, 21.3; HRMS m/z calculated for C₂₃H₂₂O₈Na 449.1201, found 449.1207.

### Compound 8

white solid (91% yield). ¹H NMR (CDCl₃, 300 MHz) δ 7.58 (s, 4H), 7.23-7.14 (m, 6H), 5.70 (m, 2H), 3.32 (m, 4H), 2.28 (s, 12H); ¹³C NMR (CDCl₃, 75 MHz) δ 169.0, 164.6, 151.1, 132.3, 132.2, 129.4, 126.9, 120.8, 120.6, 71.0, 32.1, 21.2; HRMS m/z calculated for C₃₂H₂₈O₁₂Na 627.1468, found 627.1473.

### Compounds 23 & 24

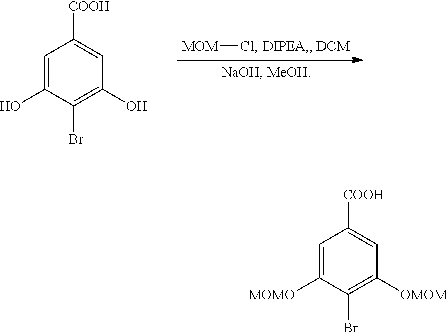

To a solution of 4-bromo, 3,5-dihydroxylbenzoic acid (500 mg, 2.1 mmol) in dry DCM at 0° C. was added diisopropylethylamine (2.49 gm, 18.9 mmol) dropwise, followed by methoxymethyl chloride (1.5 gm, 18.9 mmol) dropwise and stirred for 48 h at room temperature. After completion of the reaction as indicated by TLC, the reaction mixture was quenched with saturated NH₄Cl sol (10 ml) and extracted twice with DCM. Removal of the solvent gave the intermediate ether ester.

To this ether ester in MeOH (80 ml) was added 15% NaOH in MeOH (80 ml) and the whole was heated at 70° C. for 3 h. The pH of the reaction was adjusted to 5-6 by addition of 6N HCl at 0° C. followed by filtration. The MOM protected benzoic acid was obtained as white solid, m.p. 172° C., (400 mg, 60% yield): ¹H NMR (400 MHz, CDCl₃) δ: 7.54 (s, 2H), 5.32 (s, 4H), 3.53 (s, 6H); ¹³C NMR (400 MHz, d₄-CH₃OH) δ: 167.3, 154.8, 130.7, 109.6, 108.5, 94.8, 55.2; ESI MS m/z: 318(M⁻¹).

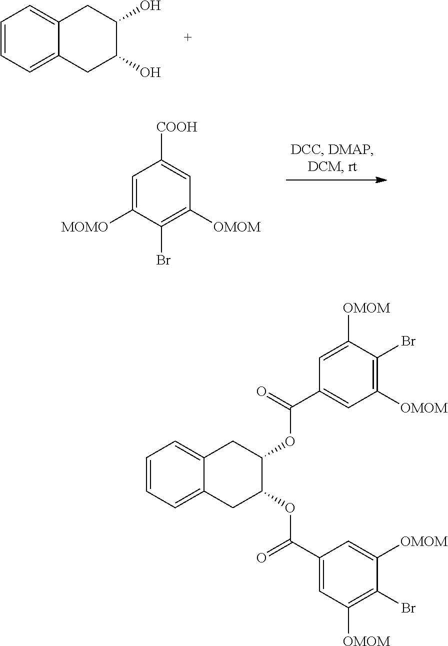

The acid (466 mg, 1.4 mmol) obtained above and dicyclohexylcarbodiimide (296 mg, 1.4 mmol) were taken in dry DCM and stirred for 1 h at rt. Then 4-dimethylaminopyridine (14 mg, 0.08 mmol) and the diol 12 (100 mg, 0.6 mmol), was added and stirred at rt. After 24 h the formed ppt was filtered off, and purified by column chromatography (3:7, Ethyl Acetate:Hexane) to give the product as clear white solid, m.p. 138° C., (400 mg, 85% yield): ¹H NMR (400 MHz, CDCl₃) δ: 7.41 (s, 4H), 7.22-7.16 (m, 4H), 5.71-5.65 (m, 2H), 5.2 (s, 8H), 3.42 (s, 12H), 3.38-3.22 (m, 4H); ¹³C NMR (400 MHz, CDCl₃) δ: 165.0, 154.8, 132.0, 130.1, 129.0, 126.5, 110.2, 109.8, 95.1, 70.6, 56.4, 32.0; ESI MS m/z: 793 (M⁺ Na).

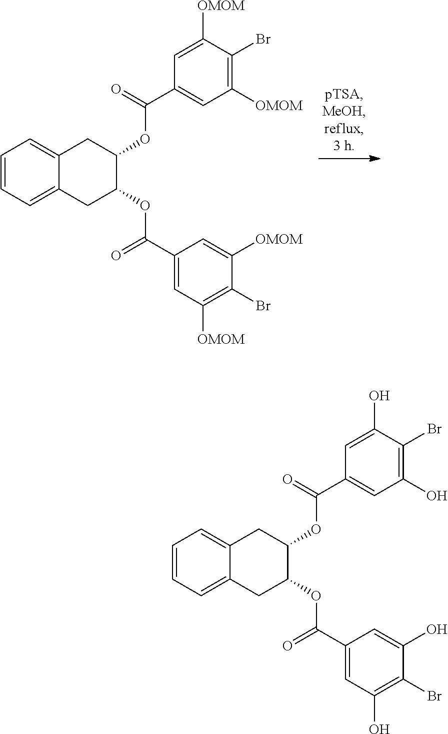

para-Toulenesulfonic acid (35 mg, 0.18 mmol) was added to a solution of the diester substrate (350 mg, 0.45 mmol) obtained above in MeOH and refluxed for 3 h. After completion of the reaction, the reaction mixture was neutralized by solid NaHCO₃, filtered and dried over NaSO₄. Purification by column chromatography gave the product 23 as white solid, m.p. 164° C., (180 mg, 65% yield): ¹H NMR (400 MHz, CDCl₃) δ: 3.4-3.2 (m, 4H), 5.75-5.6 (m, 2H), 7.11 (s, 4H), 7.20 (s, 4H), 9.13 (brs, 4H); ¹³C NMR (400 MHz, d₃-CH₃Cl) δ: 205.4, 164.8, 155.3, 132.4 130.2, 129.0, 126.4, 107.8, 70.2, 31.6; ESI MS m/z: 594 (M).

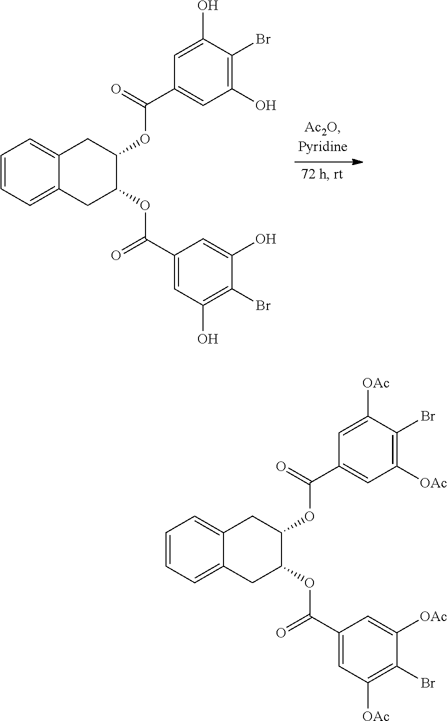

To compound 23 (80 mg, 0.16 mmol) was added acetic anhydride (1 ml) and pyridine (1 ml) and stirred for 3 days at rt. After completion of the reaction, the reaction mixture was diluted with ethyl acetate and washed with 1N HCl (10 ml), aqueous CuSO₄ solution (10 ml×3), brine (10 ml×3) and dried over Na₂SO₄. Purification by column chromatography gave compound 24 as white solid, m.p. 158° C., (80 mg, 62% yield): ¹H NMR (400 MHz, CDCl₃) δ: 2.33 (s, 12H), 3.41-3.35 (m, 4H), 5.8-5.7 (m, 2H), 7.2 (s, 4H), 7.7 (s, 4H); ¹³C NMR (400 MHz, d₆-Acetone) δ: 205.2, 167.6, 163.6, 149.7, 132.2, 130.8, 129.0, 126.4, 121.9, 117.3, 71.0, 31.3, 19.7; ESI MS m/z: 785 (M⁺ 23).

### Compounds 25 & 26

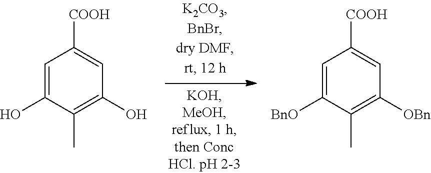

4-Methyl-3,5-dihydroxybenzoic acid (1 gm, 5.9 mmol), K₂CO₃ (2.9 gm, 21 mmol), and BnBr (3.12 gm, 18.2 mmoll) were dissolved in dry DMF and stirred for 12 h. Water was added to the reaction mixture and extracted with EtOAc thrice. The combined organic phase was evaporated and dissolved in 8N KOH in MeOH (50 ml) and refluxed for another 1 h. After completion of the reaction, the mixture was acidified with cone. HCl to pH 2-3. The formed ppt was filtered, dissolved in EtOAc and washed with water, brine and dried over NaSO₄. Removal of the solvent gave crude benzyloxybenzoic acid which on passing through small pad of celite with ethyl acetate gave pure product as white solid, m. p. 220° C. (1.4 g, 70% yield): ¹H NMR (400 MHz, d₆-Acetone) δ: 7.55-7.32 (m, 12H), 5.22 (s, 4H), 2.23 (s, 3H); ¹³C NMR (500 MHz, d₆-Acetone) δ: 166.5, 157.2, 137.4, 128.9, 128.4, 127.7, 127.3, 120.3, 106.3, 70.0, 8.4; ESI MS m/z: 347 (M−H).

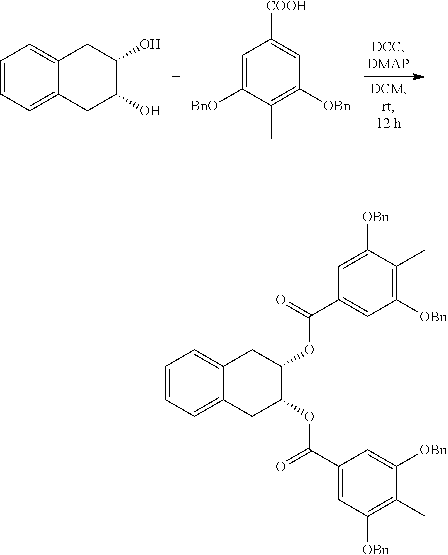

The diol 12 (50 mg, 0.3 mmol), the acid (217 mg, 0.61 mmol) obtained above, dicyclohexylcarbodiimide (263 mg, 0.61 mmol) and 4-dimethylaminopyridine (18 mg, 0.07 mmol) were dissolved in dry DCM and stirred at rt for 12 h. The DCM was removed and EtOAc was added and kept in the freezer for 12 h. The formed ppt was filtered off and the crude product was purified by column chromatography to give product as white solid, m.p. 78° C., (120 mg, 48% yield): ¹H NMR (400 MHz, CDCl₃) δ: 2.16 (s, 6H), 3.2-3.4 (dt, 4H), 4.88 (q, 8H), 5.71 (t, 2H), 7.2-7.32 (m, 28H); ¹³C NMR (500 MHz, CDCl₃) δ: 165.8, 157.1, 136.8, 132.3, 129.0, 128.4, 128.0, 127.8, 127.2, 126.6, 121.6, 106.2, 70.1, 70.0, 32.1, 9.1; ESI MS m/z: 847 (M+Na).

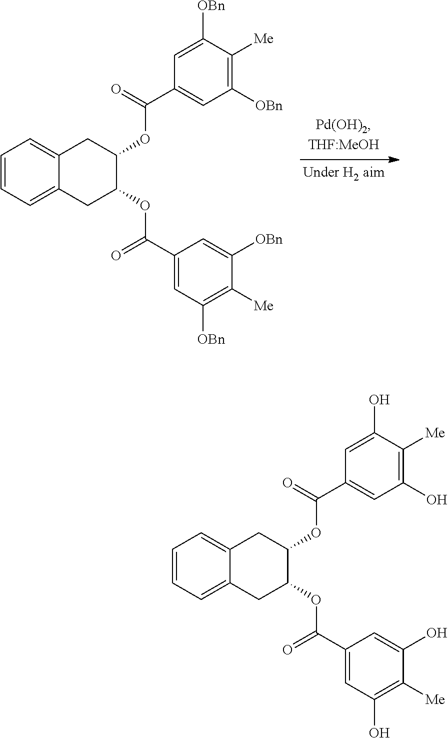

Pd(OH)₂ (60 mg, 20 wt %) was added to a solution of substrate (300 mg, 0.3 mmol) in THF:MeOH (1:2, 6 ml), and the reaction mixture was stirred at rt for 3 h. After complete conversion of the starting material into product, the Pd(OH)₂ was filtered off and the solvent was removed to give the product 25 as white solid, m.p. 142° C., (166 mg, 99% yield): ¹H NMR (500 MHz, d₆-Acetone) δ: 8.42 (s, 4H), 7.14 (s, 4H), 7.0 (s, 4H), 5.6 (t, 2H), 3.38-3.22 (m, 4H), 2.07 (s, 6H); ¹³C NMR (500 MHz, d₆-Acetone) δ: 165.4, 156.1, 132.6, 128.9, 128.2, 126.3, 116.7, 107.5, 69.8, 31.7, 8.0; ESI MS m/z: 463 (M−H).

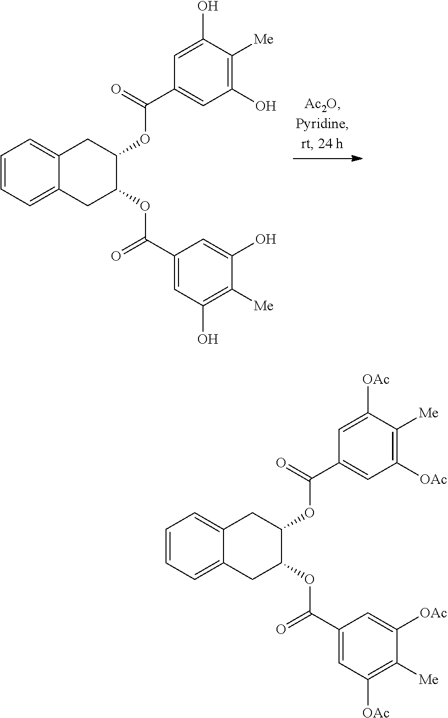

To compound 25 (50 mg, 0.1 mmol) was added acetic anhydride (1 ml) and pyridine (1 ml) and stirred for 24 h at rt. After completion of the reaction, the reaction mixture was diluted with ethyl acetate and washed with 1N HCl (10 ml), CuSO₄, (10 ml×3), brine (10 ml×3) and dried over Na₂SO₄. Purification by column chromatography gave compound 26 as white solid, m.p. 110° C., (60 mg, 88% yield): ¹H NMR (400 MHz, CDCl₃) δ: 7.53 (s, 4H), 7.25-7.15 (m, 4H), 5.66 (t, 2H), 3.4-3.29 (m, 4H), 2.31 (s, 12H), 2.01 (s, 6H); ¹³C NMR (500 MHz, d₃-CH₃Cl) δ: 168.6, 164.4, 149.8, 132.1, 129.5, 129.1, 128.9, 126.5, 121.0, 70.6, 31.9, 20.6, 10.4; ESI MS m/z: 655 (M+Na).

### Compounds 27 & 28

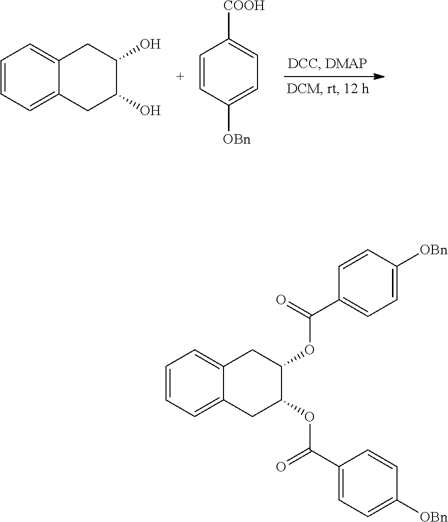

Diol 12 (50 mg, 0.3 mmol), 4-benzyloxybenzoic acid (291 mg, 0.61 mmol), dicyclohexylcarbodiimide (263 mg, 0.61 mmol) and 4-dimethylaminopyridine (9 mg, 0.07 mmol) were dissolved in dry DCM and stirred at rt for 12 h. The DCM was removed and EtOAc was added and kept in the freezer for 12 h. The formed ppt was filtered off and the crude product was purified by column chromatography to give product as white solid, m.p. 134° C.,) (115 mg, 64% yield): ¹H NMR (400 MHz, CDCl₃) δ: 3.23-3.4 (m, 4H), 5.09 (2, 4H), 5.67 (t, 2H), 6.93 (d, 4H), 7.14-7.41 (m, 14H), 7.92 (d, 4H); ¹³C NMR (500 MHz, CDCl₃) δ: 165.6, 162.5, 136.1, 132.5, 131.7, 129.1, 128.6, 128.2, 127.4, 126.4, 122.7, 114.4, 70.0, 69.9, 32.2; ESI MS m/z: 607 (M+Na).

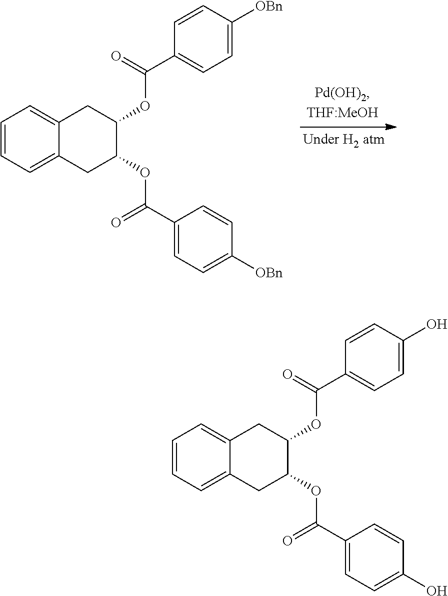

Pd(OH)₂ (40 mg, 20 wt %) was added to a solution of the substrate (230 mg, 0.3 mmol) obtained above in THF:MeOH (1:2, 6 ml), and the reaction mixture was stirred at rt for 3 h. After complete conversion of the starting material into product, the Pd(OH)₂ was filtered off and the solvent was removed to give compound 27 as white solid, m. p. 142° C., (155 mg, 98% yield): ¹H NMR (400 MHz, d₆-Acetone) δ: 7.82 (d, 4H), 7.22-7.17 (m, 4H), 6.84 (d, 4H), 5.65 (t, 2H), 3.32 (dt, 4H); ¹³C NMR (500 MHz, d₆-Acetone) δ: 165.2, 162.6, 132.8, 131.6, 129, 126.2, 120.9, 115.2, 69.6, 31.9; ESI MS m/z: 403 (M−H).

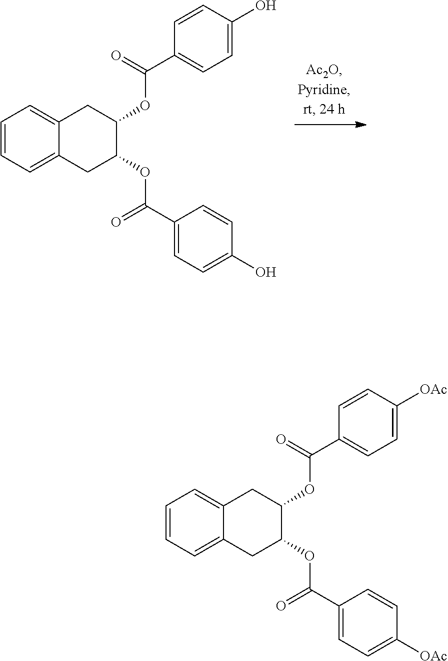

To compound 27 (40 mg, 0.09 mmol) was added acetic anhydride (1 ml) and pyridine (1 ml) and stirred for 24 h at rt. After completion of the reaction, the reaction mixture was diluted with ethyl acetate and washed with 1N HCl (10 ml), CuSO₄, (10 ml×3), brine (10 ml×3) and dried over Na₂SO₄. Purification by column chromatography gave compound 28 as white solid, m. p. 150° C., (42 mg, 88% yield): ¹H NMR (400 MHz, CDCl₃) δ: 8.01 (d, 4H), 7.2-7.11 (m, 8H), 5.7 (t, 2H), 3.33 (4H), 2.3 (s, 6H); ¹³C NMR (500 MHz, CDCl₃) δ: 168.8, 165.1, 154.4, 132.2, 131.2, 129.1, 127.6, 126.5, 121.6, 70.3, 32.1, 21.1; ESI MS m/z: 511 (M+Na).

### Compounds 29, 30 & 31

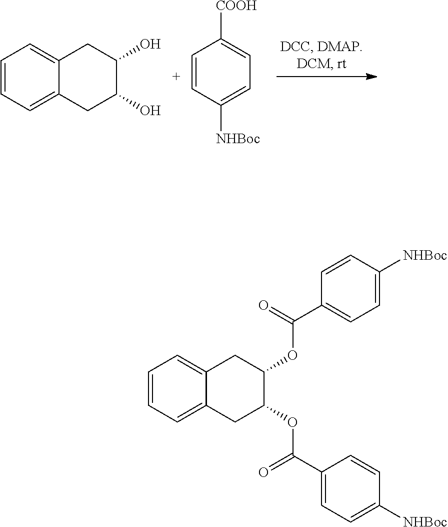

The N-boc protected acid (45 mg, 0.21 mmol) and dicyclohexylcarbodiimide (42 mg, 0.21 mmol) were taken in dry DCM and stirred for 2 h at rt, 4-Dimethylaminopyridine (3 mg, 0.03 mmol) and the diol 12 (20 mg, 0.12 mmol) were added and stirred at it. After 24 h the formed ppt was filtered off, and purified by column chromatography (1.5:8.5, Ethyl Acetate:Hexane) to give compound 29 as white solid, m.p. 110° C., (45 mg, 60% yield): ¹H NMR (400 MHz, CDCl₃) δ: 1.47 (s, 18H), 3.4-3.3.35 (m, 4H), 5.8-5.65 (m, 2H), 7.2 (s, 4H), 7.63 (d, 4H), 7.90 (d, 4H), 8.78 (brs, 2H): ¹³C NMR (400 MHz, d6-Acetone) δ: 205.3, 165.0, 152.4, 144.2, 132.7, 130.5, 129.0, 126.3, 123.7, 117.2, 117.1, 79.8, 69.8, 31.8, 27.5; ESI MS m/z: 625 (M+Na).

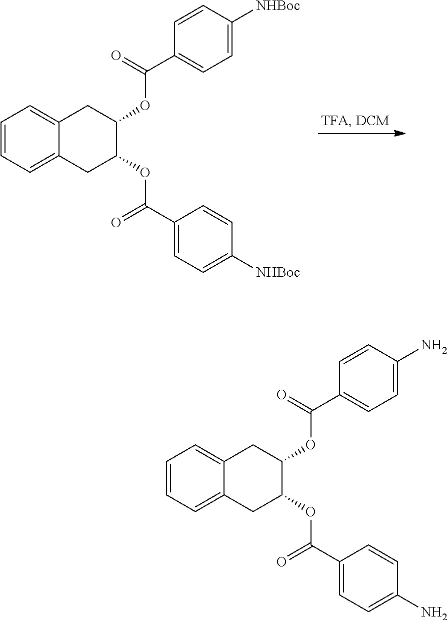

Compound 29 (130 mg, 0.2 mmol) was dissolved in DCM and a little excess of trifluoroacetic acid (246 mg, 2 mmol) was added and stirred at rt for overnight. The excess of trifluoroacetic acid was removed and the crude product was purified by column chromatography to give compound 30 as white solid, m.p. 94° C., (80 mg, 92% yield): ¹H NMR (300 MHz, d₆-Acetone) δ: 3.29-3.36 (m, 4H), 5.58-5.62 (m 2H), 6.62 (d, 4H), 6.76 (d, 1/2H), 7.17 (s, 4H), 7.69 (d, 4H), 7.92 (d, 1/2H); ¹³C NMR (400 MHz, d6-Acetone) δ: 205.3, 165.4, 132.9, 131.3, 128.9, 126.1, 117.6, 112.8, 69.2, 32.0; ESI MS m/z: 425 (M+Na).

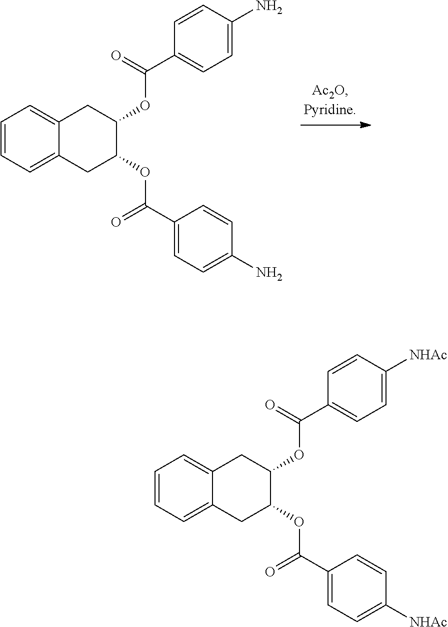

Compound 30 (40 mg, 0.09 mmol), acetic anhydride (0.5 ml) and pyridine (0.5 ml) were stirred at rt for 24 h. After completion of the reaction, ethyl acetate was added and stirred for 5 min then 1N HCL (1 ml) was added and stirred for another 5 min. The solution was washed with CuSO₄ solution (2×10 ml), water (2×10 ml), brine (2×10 ml), dried over Na₂SO₄ and purified by column chromatography to give compound 31 as white solid, m. p. 128° C., (40 mg, 82% yield): ¹H NMR (300 MHz, d₂-DCM) δ: 2.14 (s, 6H), 3.34-3.24 (m, 4H), 5.5.72-5.62 (m, 2H), 7.18 (s, 4H), 7.55 (d, 4H), 7.90 (d, 6H); ¹³C NMR (300 MHz, d₂-DCM) δ: 169.7, 165.5, 142.9, 132.4, 130.6, 129.0, 126.3, 124.9, 118.7, 70.0, 31.9, 24.0; ESI MS m/z: 509 (M+Na).

### Compound 32

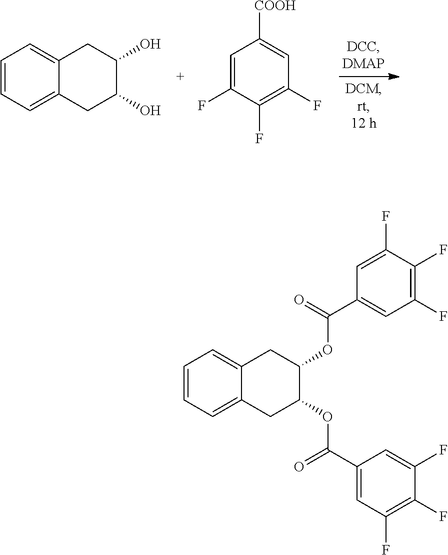

The diol 12 (50 mg, 0.3 mmol), 3,4,5-trifluorobenzoic acid (109 mg, 0.61 mmol), DCC (128 mg, 0.61 mmol) and DMAP (7 mg, 0.07 mmol) were dissolved in dry DCM and stirred at rt for 12 h. The DCM was removed and EtOAc was added and kept in the freezer for 12 h. The formed ppt was filtered off and the crude product was purified by column chromatography to give product as white solid (80 mg, 54% yield). ¹H NMR (400 MHz, CDCl₃) δ: 3.4-3.24 (m, 4H), 5.70 (t, 2H), 7.30-7.18 (m, 14H), 7.65-7.55 (m, 4H); ¹³C NMR (300 MHz, CDCl₃) δ: 31.8, 71.0, 77.4, 114.1, 114.2, 114.3, 114.4, 125.6, 125.7, 126.8, 129.1, 131.5, 141.5, 141.7, 144.8, 145.0, 145.2, 149.2, 149.3, 149.4, 152.5, 152.6, 152.7, 152.7, 163.2.

### Compound 33

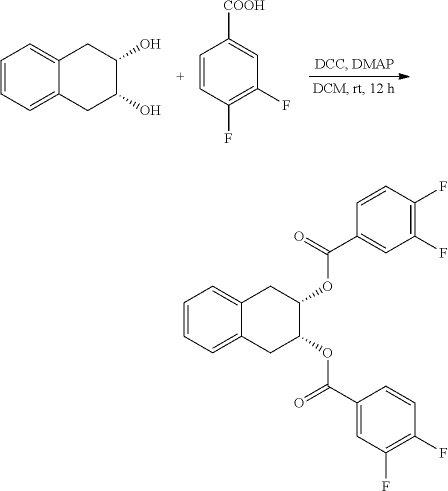

The diol 12 (50 mg, 0.3 mmol), 3,4-difluorobenzoic acid (101 mg, 0.61 mmol), DCC (131 mg, 0.61 mmol) and DMAP (7 mg, 0.07 mmol) were dissolved in dry DCM and stirred at rt for 12 h. The DCM was removed and EtOAc was added and kept in the freezer for 12 h. The formed ppt was filtered off and the crude product was purified by column chromatography to give product as white solid (91 mg, 66% yield). ¹H NMR (400 MHz, CDCl₃) δ: 3.4-3.3 (m, 4H), 5.71 (t, 2H), 7.28-7.15 (m, 6H), 7.80-7.70 (m, 4H).

### Compound 34

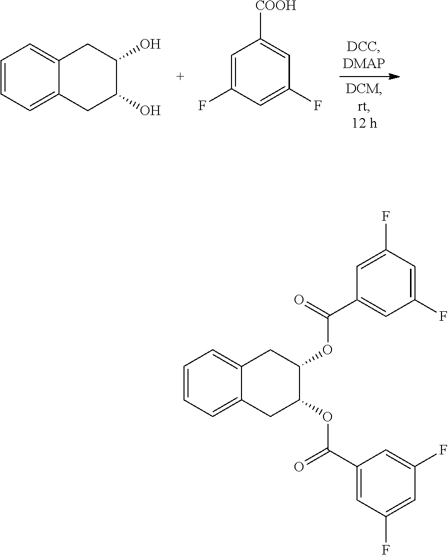

The diol 12 (50 mg, 3 mmol), 3,5-difluorobenzoic acid (101 mg, 0.61 mmol), DCC (131 mg, 0.61 mmol) and DMAP (7 mg, 0.07 mmol) were dissolved in dry DCM and stirred at rt for 12 h. The DCM was removed and EtOAc was added and kept in the freezer for 12 h. The formed ppt was filtered off and the crude product was purified by column chromatography to give product as white solid (91 mg, 66% yield). ¹H NMR (400 MHz, CDCl₃) δ: 3.4-3.30 (m, 4H), 5.72 (t, 2H), 7.02-6.98 (m, 2H) 7.30-7.18 (m, 4H), 7.47-7.40 (m, 4H).

Inhibition of purified 20S proteasome activity by EGCG and its analogs. A purified rabbit 20S proteasome (35 ng) was incubated with 20 μM of substrate Suc-LLVY-AMC in 100 μl assay buffer (20 mM Tris-HCl, pH 7.5), in the presence of EGCG or EGCG analogs at different concentrations or the solvent for 2 h at 37° C., followed by measurement of hydrolysis of the fluorogenic substrates using a Wallac Victor3™ multi-label counter with 355-nm excitation and 460-nm emission wavelengths.

Inhibition of cellular proteasome by EGCG and its analogs. Human breast cancer MDA-MB-231 cells were treated with compound 5 or 7 for 24 hours. Cell lysates were subjected to chymotrypsin activity assay and Western blotting analysis as described before.

MTT assay. Cells were grown in a 96-well plate. Triplicate wells of cells were treated with indicated concentrations of EGCG or EGCG analogs for 24 h. After aspiration of medium, MTT (1 mg/ml) was then added to the cell cultures, followed by incubation for 3 h at 37° C. After cells were crystallized, MTT was removed and DMSO was added to dissolve the metabolized MTT product. The absorbance was then measured on a Wallac Victor3 1420 Multi-label counter at 540 nm.

### Example 2

### Inhibition of Chymotrypsin-Like Activity of Purified 20S Proteasome

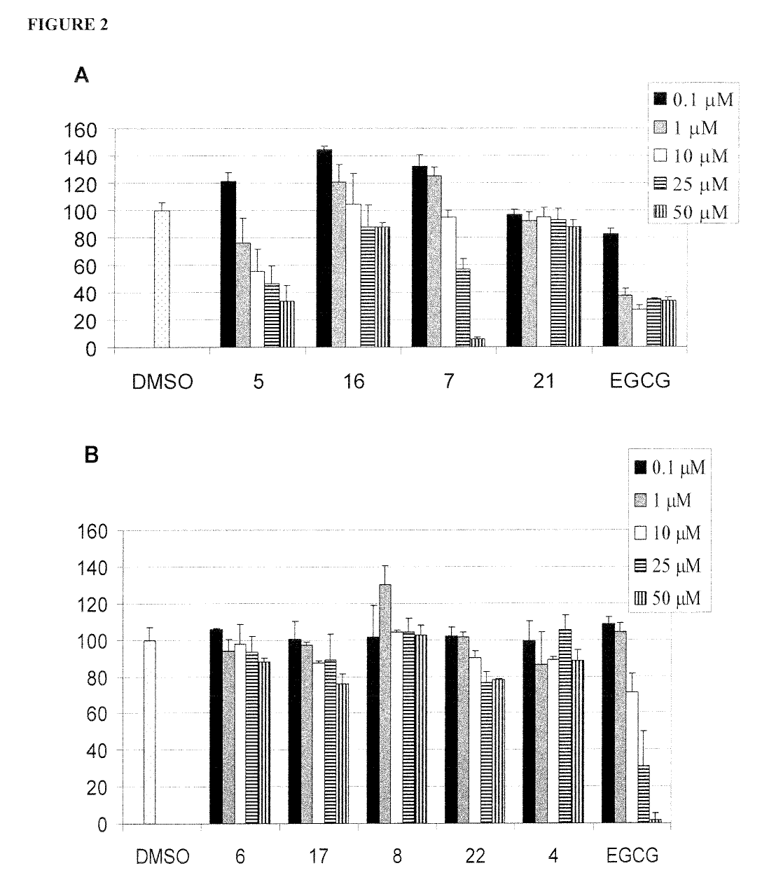

With reference to FIG. 2, EGCG potently inhibited the proteasomal chymotryptic activity consistent with our previous observation. Compound 5, which is a substituted tetralin that can be viewed as an analog of EGCG, inhibited the proteasomal chymotrypsin-like activity with an IC₅₀ value of 19 μM. Compound 16, which lacks a gallate moiety did not inhibit proteasomal chymotryptic activity even at a concentration of 50 μM. On the other hand, compound 7 (IC₅₀=29 μM) is only modestly less active than EGCG or 5 even though it lacks the gallate ester. Not surprisingly, compound 21 is not active in proteasome inhibition even at 50 μM. Upon acetylation none of the resulting derivatives 4, 6, 8, 17 and 22 exhibited proteasomal inhibition under these conditions.

### Example 3

### COMT Influences the Proteasome Inhibitory Activity of Derivatives 5 and 7

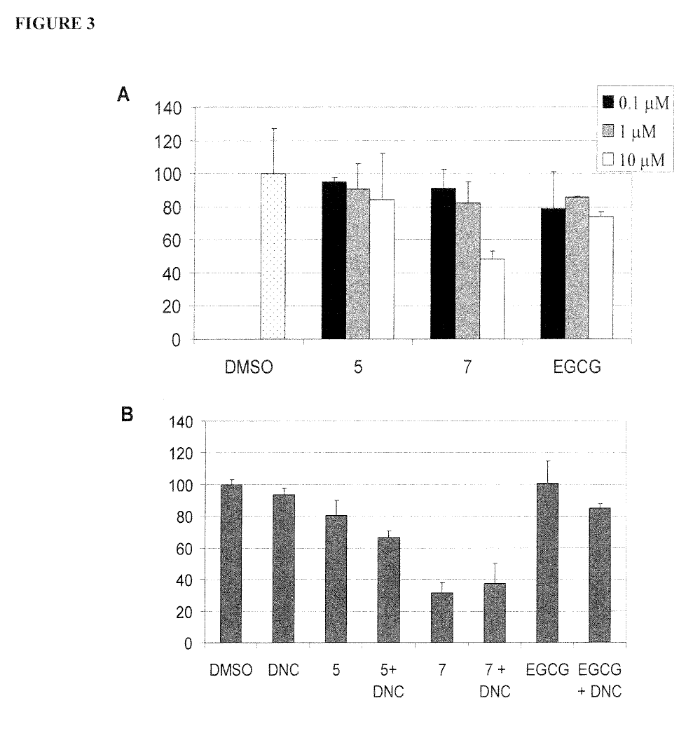

Human breast cancer MDA-MB-231 cell lysate that contains high COMT activity were treated with varying concentrations of compound 5 or 7. FIG. 3 illustrates that compound 7 at concentrations ranging between 1-10 μM inhibited proteasomal activity between 18-51% while compound 5 only inhibited proteasomal activity 10-16% under the same conditions. It would not have been expected from the data of EXAMPLE 2 that compound 7 is more active than compound 5 in inhibiting the proteasomal activity of MDA-MB-231 cell lysates. These results indicate that compound 5 may be more susceptible to biotransformation by COMT compared to compound 7. By comparison EGCG at 10 μM only inhibited the chymotrypsin-like activity in these cells by approximately 22%. Thus, consistent with previous reports EGCG is also susceptible to methylation by COMT (H., Lu, X. Meng, C. S. Yang, Drug Metabolism and Disposition; 31; 572, 2003).

### Example 4

### Inhibition of MDA-MB-231 Tumor Cell Growth

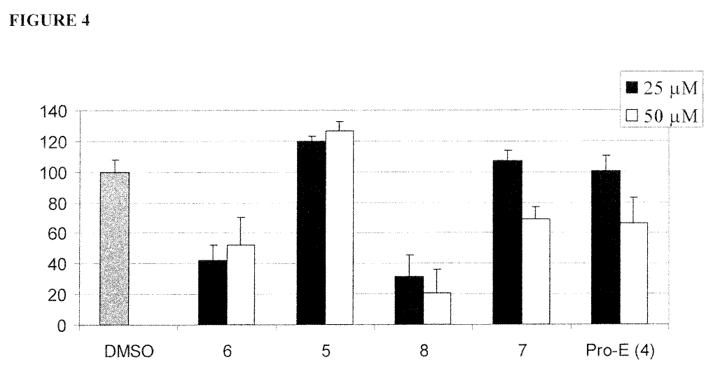

Compound 4 designated here as pro-EGCG exhibits enhanced growth inhibitory activity compared to EGCG (1) in a number of cancer cell lines (Lam, W. H. et al., Bioorg. Med. Chem. 2004, 12, 5587; Landis-Piwowar, K. R. et al., Internat. J. Mol. Med. 2005, 15, 735). It has now been determined that the per-acetates 6 and 8 are more potent in inhibiting cell growth compared to their non-acetylated precursors 5 and 7. FIG. 4 shows the growth inhibitory activity of compound 4 compared to analogs 5 and 7 and their corresponding peracetates 6 and 8 in human breast cancer MDA-MB-231 cells. Surprisingly the per acetylated analog 8 was the most potent analog, exhibiting 70-79% inhibition in MDA-MB-231 cells growth at 25 to 50 μM. The per acetylated analog 6 induced about 50% inhibition in MDA-MB-231 cells Both per acetylated analogs were more potent than pro-EGCG 4 which showed 0 to 32% inhibition at equimolar concentrations.

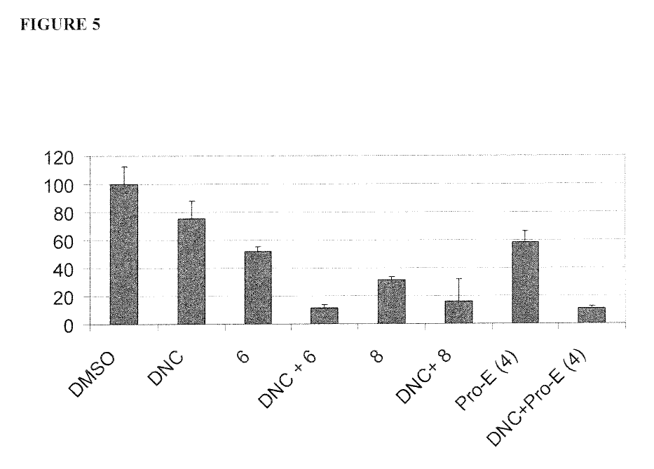

To determine whether the enhanced activity of the per acetylated analog 8 is due to diminished biotransformation, we examined whether the inhibitory activity of compounds 8 (the per-acetylated analog of 7) or 6 (the per-acetylated analog of 5) as well as that of pro-EGCG 4 is affected in the presence of 3,5-dinitrocatechol (DNC), a tight-binding inhibitor of COMT. If DNC inhibits COMT-mediated methylation of compound 5 or EGCG, one skilled in the art would observe increased growth-inhibitory activity of compound 6 or pro-EGCG on the addition of DNC. On the other hand, the growth inhibitory activity of compound 8 would not be significantly affected in the presence of DNC if analog 7 were not a substrate of COMT or would be less susceptible to its activity. MDA-MB-231 cells were treated with compounds 8 or 6, the per-acetylated analogs of compounds 7 or 5, in the presence or absence of DNC. Compound 6 alone at 50 μM inhibited cell proliferation by 48%. In the presence of 10 μM DNC the inhibition of cell proliferation mediated by analog 6 increased to 88% (FIG. 5). A similar effect was observed with Pro-EGCG 4 whose inhibition of cell proliferations increased from 42% to 89% inhibition in the presence of DNC. In contrast to analog 6 and Pro-EGCG (4), the inhibition of cell proliferation mediated by analog 8 was not greatly enhanced in the presence of DNC (69% versus 84% inhibition) Thus the compound of the invention that lacks the chatechol unit on each of the adjacent aromatic rings is as susceptible to methylation mediated by COMT, which manifests higher inhibition of cell growth proliferation.

### Example 5

### Accumulation of Ubiquitinated Proteins

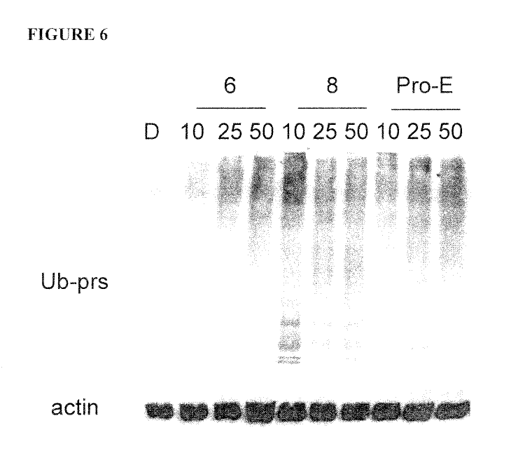

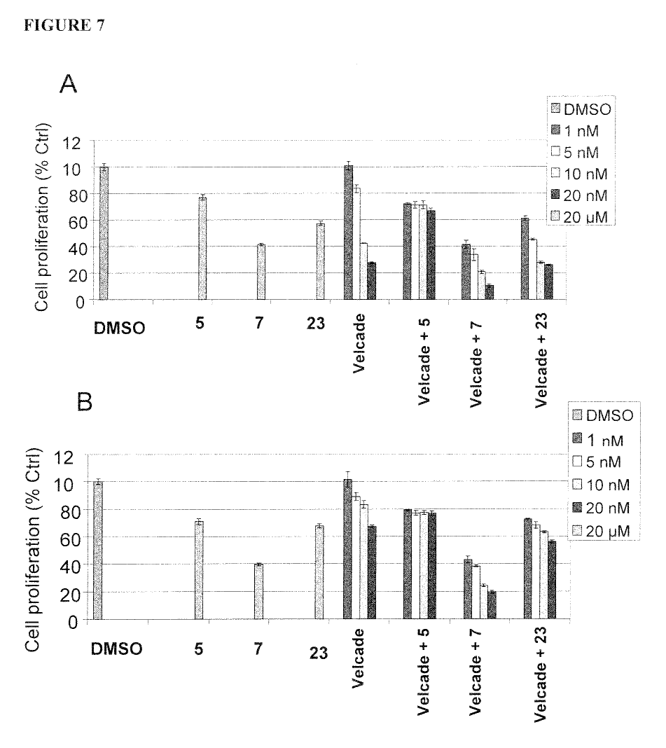

With reference to FIG. 6, the experiments of this example 5 were undertaken to investigate whether in MDA-MB-231 cells pre-acetylated compounds 6 and compound 8 as well as Pro-EGCG 4 would be able to inhibit proteasome and manifest accumulation of ubiquitinated proteins. Indeed, at the doses tested and illustrated in FIG. 7, higher levels of ubiquitinated proteins were accumulated by compound 8 compared to compound 6 and Pro-EGCG 4 in MDA-MB-231 cells, indicating that more proteasome activity was inhibited by analog 8.

### Example 6

### Inhibition of Cell Proliferation of Human Multiple Myeloma Cells by Compounds of the Invention in Combination with Bortezomib

When combined with bortezomib (Velcade™), compounds 7 and 23 but not 5 showed synergistic inhibitory effect against cell proliferation in human multiple myeloma cells. Among the three analogs, compound 7 was the most potent cell proliferation inhibitor. Cell proliferation was inhibited ˜60% in ARP cells treated with 20 μM of compound 7 (FIG. 7A). Treatment with bortezomib reduced cell proliferation in a dose-dependent manner and further inhibition was observed when combined with compound 7 (FIG. 7A). The inhibitory effect of bortezomib was interfered with by co-treatment with compound 5 (FIG. 7A). The compound 23 also increased bortezomib-induced inhibition of cell proliferation but the effect was weaker than that seen with compound 7 (FIG. 7A).

The data generated from OPM1 cells showed a similar pattern of effects. However the OPM1 cell line appears to be more resistant to treatment with bortezomib and the combinations (FIG. 7B).

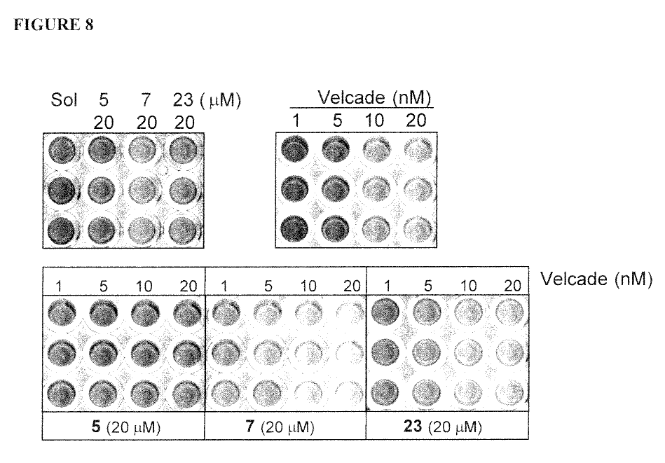

In FIG. 8, color changes of MTT assay in a 96 well-plate (in the same experiment shown in FIG. 7A) using ARP cells are presented. Deep purple color indicates fully viable cells; light purple color indicates a reduced number of viable cells; and yellowish color indicates an absence of viable cells (FIG. 8). The color change pattern was consistent with the potencies of the compounds for inhibiting ARP tumor cell growth (compare FIGS. 8 and 7A). The results given in FIGS. 7 and 8 show that compounds 7 and 23 unexpectedly exhibit synergistic effect on human multiple myeloma cells when combined with bortezomib, and compound 5 could partially block the inhibitory effect of bortezomib on the malignant cells.

While specific embodiments of the present invention have been described in the examples, it is apparent that modifications and adaptations of the present invention will occur to those skilled in the art. The embodiments of the present invention are not intended to be restricted by the examples. It is to be expressly understood that such modifications and adaptations which will occur to those skilled in the art are within the scope of the present invention, as set forth in the following claims. For instance, features illustrated or described as part of one embodiment can be used in another embodiment, to yield a still further embodiment. Thus, it is intended that the present invention cover such modifications and variations as come within the scope of the claims and their equivalents.

The contents of all documents and references cited herein are hereby incorporated by reference in their entirety.

1. A compound having the structure of formula I: 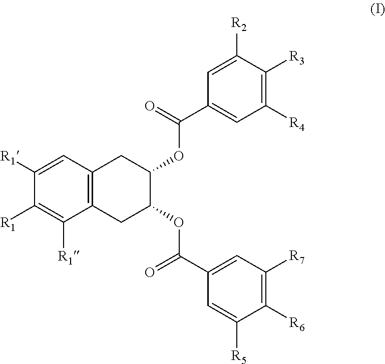
wherein R₁, R₁′ and R₁″ are each independently selected from the group consisting of H, alkyl, alkenyl, cycloalkyl, heterocycloalkyl, cycloalkenyl, heterocycloalkenyl, aryl, halogen, OH, an acyloxy group, and NR₈,R₉, wherein R₈ and R₉ are independently selected from the group consisting of H, alkyl, alkenyl, cycloalkyl, heterocycloalkyl, cycloalkenyl, heterocycloalkenyl, aryl, and acyl, any of which may be optionally substituted;
R₂, R₄, R₅ and R₇ are each independently H, alkyl, alkenyl, cycloalkyl, heterocycloalkyl, cycloalkenyl, heterocycloalkenyl, aryl, OH, acyloxy or halogen; and
R₃ and R₆ are each independently H, OH, acyloxy, NR₈R₉ or a halogen, wherein R₈ and R₉ are as defined above;
or an analog thereof;
or a pharmaceutically acceptable salt thereof.

2. The compound of claim 1, wherein the compound has the structure of formula Ia: 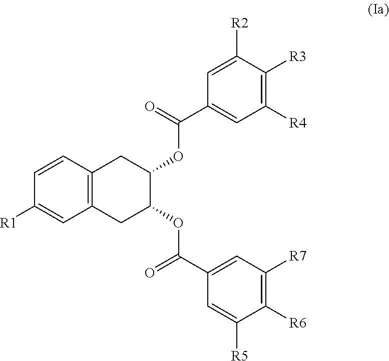
wherein R₁ is selected from the group consisting of H, alkyl, alkenyl, cycloalkyl, heterocycloalkyl, cycloalkenyl, heterocycloalkenyl, aryl, halogen, OH, an acyloxy group, and NR₈,R₉, wherein R₈ and R₉ are independently selected from the group consisting of H, alkyl, alkenyl, cycloalkyl, heterocycloalkyl, cycloalkenyl, heterocycloalkenyl, aryl, and acyl, any of which may be optionally substituted;
R₂, R₄, R₅ and R₇ are each independently H, alkyl, alkenyl, cycloalkyl, heterocycloalkyl, cycloalkenyl, heterocycloalkenyl, aryl, OH, acyloxy or halogen; and
R₃ and R₆ are each independently H, OH, acyloxy, NR₈R₉ or halogen;
or an analog thereof;
or a pharmaceutically acceptable salt thereof.

3. The compound of claim 1, wherein:
R₁ is selected from the group consisting of H, halogen, OH, and an acyloxy group; R₂, R₄, R₅ and R₇ are each independently H, alkyl, alkenyl, cycloalkyl, heterocycloalkyl, cycloalkenyl, heterocycloalkenyl, aryl, OH, acyloxy or halogen; and R₃ and R₆ are each independently H, OH, acyloxy, NR₈R₉ or a halogen.

4. A compound having the structure of formula II: 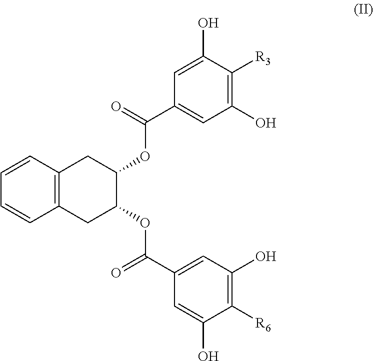
wherein:
R₃ and R₆ are both H, Br, F, Cl or CH₃;
or an analog thereof;
or a pharmaceutically acceptable salt thereof; or
a compound having the structure of formula III: 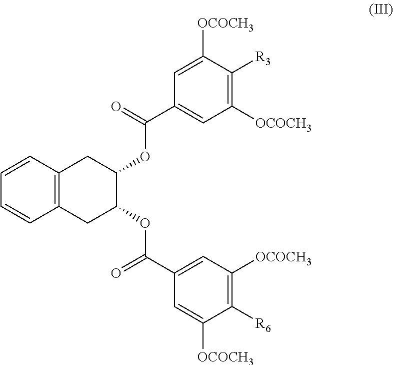
wherein:
R₃ and R₆ are both OCOCH₃, H, Br, F, Cl or CH₃;
or an analog thereof;
or a pharmaceutically acceptable salt thereof; or
a compound having the structure of formula IV: 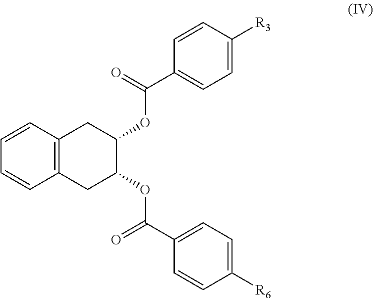
wherein:
R₃ and R₆ are both OH, OCOCH₃, NHCOOC(CH₃)₃, NH₂ or NHCOCH₃;
or an analog thereof;
or a pharmaceutically acceptable salt thereof; or
a compound having the structure of formula VII: 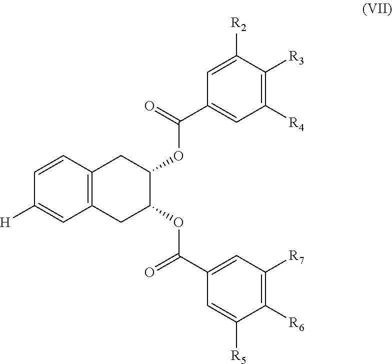
wherein R₂, R₃, R₄, R₅, R₆ and R₇ are F; or
R₂, R₃, R₅ and R₆ are F, and R₄ and R₇ are H; or
R₂, R₄, R₅ and R₇ are F, and R₃ and R₆ are H;
or an analog thereof;
or a pharmaceutically acceptable salt thereof.

5. A compound having the following structure: 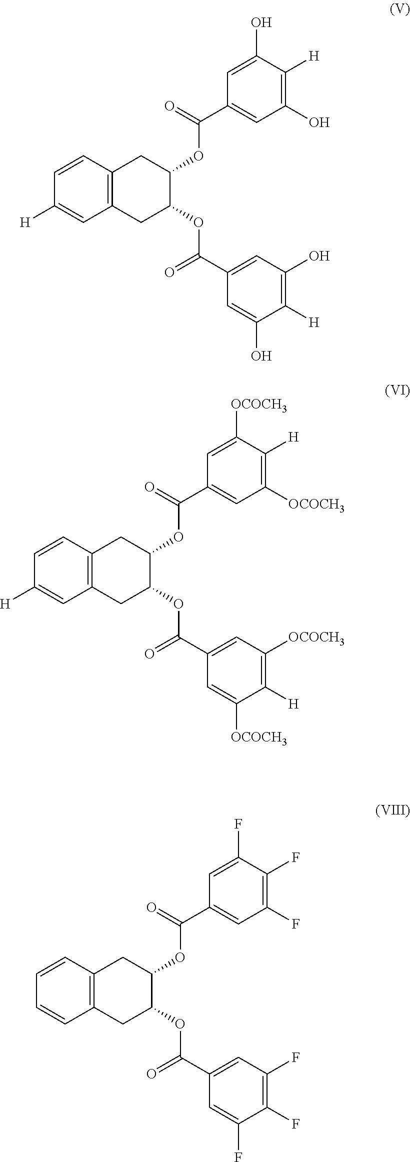 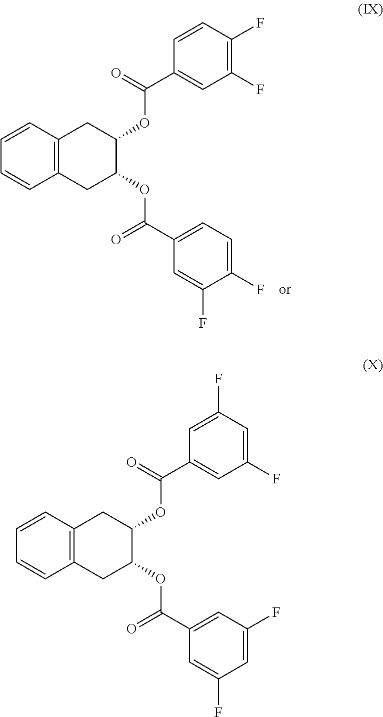
or an analog thereof;
or a pharmaceutically acceptable salt thereof; or
a compound having a structure selected from the group consisting of the structures shown in Table 1, the structures shown in Scheme 1, the structures shown in Scheme 2, the structures shown in Scheme 3, and analogs thereof; or a pharmaceutically acceptable salt thereof.

6. A pharmaceutical composition comprising at least one compound as defined in claim 1 and a pharmaceutically acceptable carrier.

7. A method for inhibiting proteasomal activity in a cell, comprising contacting the cell with an effective amount of at least one compound as defined in claim 1 or a pharmaceutical composition thereof, such that proteasomal activity in the cell is inhibited.

8. The method according to claim 7, wherein said contacting occurs in vitro.

9. The method according to claim 7, wherein said contacting occurs in vivo.

10. The method according to claim 9, wherein said contacting comprises administering the at least one compound or the composition to a subject by a route selected from the group consisting of oral, parenteral, subcutaneous, intravenous, intramuscular, intraperitoneal, intraarterial, transdermal, and mucosal administration.

11. The method according to claim 7, wherein said proteasome is a 20S proteasome or a 26S proteasome.

12. The method according to claim 7, wherein said proteasome is a 20S proteasome and the chymotrypsin activity and/or the chymotrypsin-like activity of the 20S proteasome is inhibited.

13. A method for treating cancer in a subject comprising administering a therapeutically effective amount of at least one compound as defined in claim 1 or a pharmaceutical composition thereof to the subject.

14. The method of claim 13, wherein cancer cell growth is inhibited in the subject.

15. The method of claim 13, wherein cancer cell apoptosis is induced in the subject.

16. The method of claim 13, wherein proteasomal activity is inhibited in the subject.

17. The method of claim 13, wherein the cancer is selected from the group consisting of prostate cancer, leukemia, hormone dependent cancers, breast cancer, colon cancer, lung cancer, epidermal cancer, liver cancer, esophageal cancer, stomach cancer, cancer of the brain, kidney cancer and multiple myeloma.

18. The method of claim 7, wherein the compound or composition is administered orally.

19. The method of claim 7, wherein the subject is a human.

20. A method for synthesizing the compound of claim 1, comprising the steps of:
(a) Reacting dihydronaphthalene with osmium tetroxide;
(b) Acylating with two or more equivalents of a suitably protected aryl benzoic acid and a dehydrating agent; and
(c) Removing the protecting group in the presence of a catalyst.

21. The method of claim 20, further comprising the step of:
(d) Reacting the compound of step (c) with an acylating agent.

22. The method of claim 20, wherein the protecting group is benzyloxy.

23. The method of claim 7, wherein the compound or composition is administered in combination with a second therapeutic agent.

24. The method of claim 23, wherein the second therapeutic agent is an anti-cancer therapeutic agent, a chemotherapeutic agent, and/or a proteasomal inhibitor.

25. The method of claim 24, wherein the second therapeutic agent is selected from the group consisting of Taxol™, docetaxel, vinblastine, vincristine, camptothecin toptecan, etoposid, teniposide, salinosporamide, epigallocatechin gallate and analogs thereof.

26. The method of claim 24, wherein the second therapeutic agent is bortezomib (Velcade™) or an analog thereof,

27. The method of claim 23, wherein the cancer is multiple myeloma.

28. The method of claim 23, wherein the compound or composition and the second agent are co-administered.

29. The method of claim 23, wherein the compound or composition and the second agent are administered sequentially.

30. A method for treating multiple myeloma comprising administering a therapeutically effective amount of a compound as defined in claim 1 to a subject in need thereof, such that multiple myeloma is treated.

31. The method of claim 30, wherein the compound or composition is administered in combination with an anti-cancer therapeutic or a chemotherapeutic agent.

32. The method of claim 30 wherein the compound or composition is administered in combination with bortezomib (Velcade™).
